(19) United States
(12) Patent Application Publication
(10) Pub. No.: US 2025/0169883 A1
(43) Pub. Date: May 29, 2025

(54) ORTHOPAEDIC ALIGNMENT GUIDES AND METHODS OF REPAIR

(71) Applicant: ARTHREX, INC., Naples, FL (US)

(72) Inventors: Nick Metcalfe, Bonita Springs, FL (US); Steven Jim DeLeon, Naples, FL (US); Scott William Doody, Bonita Springs, FL (US); David Knopf, Bonita Springs, FL (US)

(21) Appl. No.: 18/842,524

(22) PCT Filed: Feb. 10, 2023
(86) PCT No.: PCT/US2023/062361

§ 371 Date: Aug. 29, 2024

Related U.S. Application Data
(60) Provisional application No. 63/315,663, filed on Mar. 2, 2022.

(51) Int. Cl. A61B 34/10; A61B 17/17; A61B 17/56

(52) U.S. Cl. CPC A61B 34/10; A61B 17/1775; A61B 2017/568; A61B 2034/107; A61B 2034/108

(57) ABSTRACT

This disclosure relates to surgical planning systems, instrumentation and methods for repairing bone defects. The planning systems and instrumentation disclosed herein may be utilized for placement of guide members to establish positions of surgical instruments. The surgical instruments may be utilized to establish resection surfaces for fusion of adjacent bone surfaces.

### BACKGROUND

This disclosure relates to surgical devices and methods for repairing bone defects along articular surfaces of a joint.

Many bones of the human musculoskeletal system include articular surfaces. The articular surfaces articulate relative to other bones to facilitate different types and degrees of joint movement. The articular surfaces can erode (e.g., experience bone loss) over time due to repeated use or wear or can fracture as a result of a traumatic impact. These types of bone defects can cause joint instability and pain.

Bone deficiencies may occur along the articular surfaces of ankle bones. Some techniques utilize a bone plate to fix the ankle bones to each other.

### SUMMARY

This disclosure relates to planning systems, alignment guides and methods of performing a surgical procedure. The planning systems, alignment guides and methods may be utilized for planning and implementing orthopaedic procedures to restore functionality to a joint. The disclosed alignment guides may be utilized for positioning one or more guide members relative to bone or other tissue.

An alignment guide for an orthopaedic procedure of the present disclosure may include a guide body that may carrying a first set of adjustable contact members dimensioned to contact bone at respective contact points. The guide body may include at least one guide passage dimensioned to set a trajectory of a guide pin relative to bone. At least one adjustment body may be coupled to the guide body along an interface. The at least one adjustment body may carry a second set of adjustable contact members dimensioned to contact bone at respective contact points. Each contact member of the second set of contact members may be coupled to a respective carrier. The carrier may be translatable along a guide shaft to set a position of the respective contact member relative to the at least one adjustment body in response to rotation of the guide shaft.

An alignment guide for an orthopaedic procedure of the present disclosure may include a guide body that may carry a first set of adjustable contact members dimensioned to contact bone at respective contact points. The guide body may include at least one guide passage dimensioned to set a trajectory of a guide pin relative to bone. A set of adjustment bodies may be interchangeably mountable to the guide body along an interface. Each of the adjustment bodies may carry a second set of adjustable contact members dimensioned to contact bone at respective contact points. The second set of contact members may be distributed in a first direction and may be moveable in a second direction relative to the respective adjustment body. The adjustment bodies may be dimensioned such that a distance between one or more adjacent pairs of the second set of contact members associated with the same anatomical points of the bone may differ within the set of adjustment bodies relative to the first direction.

A method of performing an orthopaedic procedure of the present disclosure may include determining a resection plane relative to an articular surface of a bone. The method may include determining an orientation of at least one guide pin relative to the resection plane. The method may include determining positions of a first set of anatomical points along a non-articular surface of the bone. The method may include determining positions of a second set of anatomical points along the articular surface of the bone. The method may include configuring an alignment guide. The alignment guide may include a guide body coupled to at least one adjustment body along an interface extending in a first direction. The guide body may include a guide passage. The guide body may carry a first set of adjustable contact members. The at least one adjustment body may carry a second set of adjustable contact members distributed in the first direction. The configuring step may include setting a position of each contact member of the first and second sets of contact members based on the determined orientation. The configuring step may include setting the position of each contact member of the of the second set of contact members based on the determined positions of the second set of anatomical points. The configuring step may include setting a position of the at least one adjustment body and the guide body relative to each other along the interface based on the determined positions of the first and second sets of anatomical points. The method may include positioning the alignment guide relative to the bone according to the set positions such that the second set of contact members contact the respective anatomical points of the second set of anatomical points, and then inserting the at least one guide pin through the guide passage and into the bone.

A method of performing an orthopaedic procedure of the present disclosure may include determining a resection plane relative to an articular surface of a bone. The bone may extend along a longitudinal axis between a proximal end and a distal end. A periphery of the bone may establish a saddle extending axially between the proximal and distal ends. The method may include determining an orientation of at least one guide pin relative to the resection plane. The method may include determining positions of a first set of anatomical points along a non-articular surface of the bone. The first set of anatomical points may be distributed on opposite sides of the saddle. The method may include determining positions of a second set of anatomical points along the articular surface of the bone. The method may include configuring an alignment guide. The alignment guide may include a guide body coupled to at least one adjustment body along an interface. The guide body may include a guide passage. The guide body may carry a first set of adjustable contact members that may be distributed on opposite sides of a reference plane that bisects the guide body. The at least one adjustment body may carry a second set of adjustable contact members. The configuring step may include setting a position of each of the first and second sets of contact members based on the determined orientation. The configuring step may include setting the position of each of the first set of contact members based on the determined positions of the first set of anatomical points. The configuring step may include setting the position of each of the second set of contact members based on the determined positions of the second set of anatomical points. The method may include positioning the alignment guide relative to the bone according to the set positions. The method may include positioning the first set of contact members in contact with the first set of anatomical points such that the reference plane is substantially parallel to the longitudinal axis of the bone. The method may include positioning the second set of contact members in contact with the second set of anatomical points. The method may include inserting the at least one guide pin through the guide passage and into the bone subsequent to the positioning of the alignment guide.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates an exemplary planning system.

FIG. 2 illustrates another exemplary planning system including a user interface.

FIG. 3 illustrates the user interface of FIG. 2 including display windows depicting adjacent bone models.

FIG. 4 illustrates an exemplary bone model.

FIG. 5 illustrates the user interface of FIG. 2 including display windows depicting landmarks relative to adjacent bone models.

FIG. 6 illustrates the user interface of FIG. 2 including display windows depicting reference planes and resection surfaces associated with the bone models.

FIG. 7 illustrates the user interface of FIG. 2 including display windows depicting an implant model positioned relative to adjacent bone models.

FIGS. 8-10 illustrate exemplary trajectories for adjacent bone models in display windows of the user interface of FIG. 2.

FIG. 11 illustrates exemplary anatomical points associated with a bone model in a display window of the user interface of FIG. 2.

FIG. 12 illustrates data points of anatomical samples of a silhouette of the articular surface of a bone of the anatomy.

FIG. 13 illustrates data points of pathological samples of a silhouette of the articular surface of a bone of the anatomy.

FIG. 14 illustrates a perspective view of an exemplary alignment guide.

FIG. 15 illustrates a plan view of the alignment guide of FIG. 14.

FIG. 16 illustrates a side view of the alignment guide along line 16-16 of FIG. 15.

FIG. 17 illustrates a sectional view of the alignment guide along line 16-16 of FIG. 15.

FIG. 18 illustrates a set of adjustment bodies associated with the alignment guide of FIG. 15.

FIG. 19 illustrates a sectional view of the alignment guide along line 19-19 of FIG. 15.

FIG. 20 illustrates a side view of the alignment guide along line 20-20 of FIG. 15.

FIGS. 21-23 illustrate implementations of a contact member positioned relative to bone.

FIG. 24 illustrates an exemplary method of planning and implementing an orthopaedic procedure.

FIG. 25 illustrates a plan view of an alignment guide including with contact members positioned relative to articular and non-articular surfaces of a bone.

FIG. 26 illustrates a perspective view of the alignment guide positioned relative to the bone of FIG. 25.

FIG. 27 illustrates a plan view of the alignment guide of FIG. 25 with carriers associated with the contact members omitted for illustrative purposes.

FIG. 28 illustrates an end view of the alignment guide positioned relative to the bone of FIG. 25.

FIG. 29 illustrates another perspective view of the alignment guide and an alignment member positioned relative to the bone of FIG. 25.

FIG. 30 illustrates a plan view of a cutting guide positioned relative to the bone of FIG. 25.

FIG. 31 illustrates an end view of the cutting guide positioned relative to the bone of FIG. 30 with a portion of the bone removed.

FIG. 32 illustrates a plan view of another cutting guide positioned relative to the bone of FIG. 31.

FIG. 33 illustrates a side view of the bone of FIG. 32 with another portion of the bone removed.

FIG. 34 illustrates perspective view of another cutting guide positioned relative to another bone adjacent to the bone of FIG. 30.

FIG. 35 illustrates another perspective view of the cutting guide positioned relative to the bone of FIG. 34 with a portion of the bone removed.

FIG. 36 illustrates a side view of an implant positioned relative to adjacent bones.

FIG. 37 illustrates a side view of the implant secured to the adjacent bones of FIG. 36.

Like reference numbers and designations in the various drawings indicate like elements.

### DETAILED DESCRIPTION

This disclosure relates to surgical devices and methods for repairing bone defects. The instrumentation and systems described herein may be capable of dimensioning or otherwise preparing a defect surface at a surgical site, including resecting bone or other tissue.

The disclosed planning systems and methods may be utilized to determine placement of guide members for resecting or otherwise removing a portion of the associated bone or other tissue, which can limit removal of tissue and improve healing. The surgeon or assistant may interact with the disclosed planning systems to set and adjust anatomical points and other landmarks associated with the selected bone models, which may be utilized to establish one or more resection planes along the bone. Aspects of a surgical plan can be established based on the parameters, including various settings and dimensions associated with instrumentation to prepare a surgical site. The disclosed alignment guides may be utilized to establish a precise orientation of guide members in a manner that substantially conforms to an associated surgical plan.

An alignment guide for an orthopaedic procedure of the present disclosure may include a guide body that may carrying a first set of adjustable contact members dimensioned to contact bone at respective contact points. The guide body may include at least one guide passage dimensioned to set a trajectory of a guide pin relative to bone. At least one adjustment body may be coupled to the guide body along an interface. The at least one adjustment body may carry a second set of adjustable contact members dimensioned to contact bone at respective contact points. Each contact member of the second set of contact members may be coupled to a respective carrier. The carrier may be translatable along a guide shaft to set a position of the respective contact member relative to the at least one adjustment body in response to rotation of the guide shaft.

In a further implementation, the second set of contact members may be distributed in a first direction along the at least one adjustment body. The at least one adjustment body may include a set of adjustment bodies. The adjustment bodies may be interchangeably mountable to the guide body along the interface. The adjustment bodies may be dimensioned such that a distance between one or more adjacent pairs of the second set of contact members associated with the same anatomical points of the bone may differ within the set of adjustment bodies relative to the first direction.

In a further implementation, the guide shaft may extend along a slot of the at least one adjustment body. The carrier may be captured in the respective slot. Axial movement of the carrier along the guide shaft may be bounded by opposed ends of the slot. The carrier may abut opposed sidewalls of the slot to limit rotation of the carrier about the guide shaft.

In a further implementation, each of the first set of contact members may be a threaded shaft coupled to the guide body at a threaded connection. Each of the first set of contact members may carry a respective sheath that may dimensioned to abut a sidewall of the guide body. The sheath may be dimensioned to set a position of an indicator relative to a ruler in response to relative movement between the threaded shaft and the guide body.

In a further implementation, the first set of contact members may be dimensioned such that the contact points may be established at a free end of the respective contact members. The second set of contact members may be dimensioned such that the contact points may be spaced apart from a free end of the respective contact members.

In a further implementation, each contact member of the first and second sets of contact members may include an elongated body extending along a respective contact axis. The contact axes of the first set of contact members may be substantially parallel to each other. The contact axes of the second set of contact members may be substantially parallel to each other.

In a further implementation, a reference plane may bisect the guide body. The first set of contact members may include first and second pairs of contact members. The first pair of contact members may be positioned on a first side of the reference plane. The second pair of the contact members can positioned on a second side of the reference plane. The reference plane may intersect the at least one adjustment body such that at least one contact member of the second set of contact members may be positioned on the first side of the reference plane and such that at least one other contact member of the second set of contact members may be positioned on the second side of the reference plane for all positions of the at least one adjustment body relative to the interface.

An alignment guide for an orthopaedic procedure of the present disclosure may include a guide body that may carry a first set of adjustable contact members dimensioned to contact bone at respective contact points. The guide body may include at least one guide passage dimensioned to set a trajectory of a guide pin relative to bone. A set of adjustment bodies may be interchangeably mountable to the guide body along an interface. Each of the adjustment bodies may carry a second set of adjustable contact members dimensioned to contact bone at respective contact points. The second set of contact members may be distributed in a first direction and may be moveable in a second direction relative to the respective adjustment body. The adjustment bodies may be dimensioned such that a distance between one or more adjacent pairs of the second set of contact members associated with the same anatomical points of the bone may differ within the set of adjustment bodies relative to the first direction.

In a further implementation, the interface may extend in the first direction.

In a further implementation, a total number of the contact members of the second set of contact members may be equal for the set of adjustment bodies.

In a further implementation, each contact member of the first and second sets of contact members may include an elongated body extending along a respective contact axis. The contact axes of the first set of contact members may be substantially parallel to each other. The contact axes of the second set of contact members may be substantially parallel to each other.

In a further implementation, each contact member of the second set of contact members may be coupled to a respective carrier. The carrier may be translatable along a respective guide shaft to set a position of the respective contact member relative to the respective adjustment body in response to rotation of the guide shaft.

In a further implementation, each contact member of the second set of contact members may extend along a contact axis between a first end portion and a second end portion. The first end portion may be coupled to the carrier at a fixed position relative to the contact axis such that the second end portion of the contact member may be cantilevered from the carrier.

In a further implementation, the carrier may be dimensioned to set a position of an indicator relative to a ruler in response to relative movement between the carrier and the respective adjustment body.

A method of performing an orthopaedic procedure of the present disclosure may include determining a resection plane relative to an articular surface of a bone. The method may include determining an orientation of at least one guide pin relative to the resection plane. The method may include determining positions of a first set of anatomical points along a non-articular surface of the bone. The method may include determining positions of a second set of anatomical points along the articular surface of the bone. The method may include configuring an alignment guide. The alignment guide may include a guide body coupled to at least one adjustment body along an interface extending in a first direction. The guide body may include a guide passage. The guide body may carry a first set of adjustable contact members. The at least one adjustment body may carry a second set of adjustable contact members distributed in the first direction. The configuring step may include setting a position of each contact member of the first and second sets of contact members based on the determined orientation. The configuring step may include setting the position of each contact member of the of the second set of contact members based on the determined positions of the second set of anatomical points. The configuring step may include setting a position of the at least one adjustment body and the guide body relative to each other along the interface based on the determined positions of the first and second sets of anatomical points. The method may include positioning the alignment guide relative to the bone according to the set positions such that the second set of contact members contact the respective anatomical points of the second set of anatomical points, and then inserting the at least one guide pin through the guide passage and into the bone.

In a further implementation, the positioning step may occur such that the first set of contact members may establish contact along respective contact points of a non-articular surface of the bone.

In a further implementation, the method includes positioning a resection guide along the at least one guide pin such that an orientation of a resection slot of the resection guide may substantially correspond to the determined resection plane. The method may include moving a cutting instrument through the resection slot to resect a portion of the bone.

In a further implementation, the bone may be a tibia. The step of determining the positions of the second set of anatomical points may include identifying the positions of the second set of anatomical points in a silhouette of the articular surface of the tibia. The second set of anatomical points may be distributed in a medial-lateral direction of the tibia.

In a further implementation, the second set of anatomical points may include a medial maximum and a tibial plafond of the articular surface of the tibia.

In a further implementation, the second set of anatomical points may include a tibial groove and a lateral maximum of the articular surface of the tibia.

In a further implementation, the step of setting the position of the at least one adjustment body may include moving the at least one adjustment body and the guide body in the first direction relative to each other along the interface.

In a further implementation, the at least one adjustment body may include a set of adjustment bodies. The adjustment bodies may be interchangeably mountable to the guide body along the interface. The adjustment bodies may be dimensioned such that a distance between one or more adjacent pairs of the second set of contact members associated with the same anatomical points of the second set of anatomical points of the bone may differ within the set of adjustment bodies relative to the first direction. The step of configuring the alignment guide may include selecting the at least one adjustment body from the set of adjustment bodies based on the determined positions of the first and second sets of anatomical points. The step of setting the position of each of the second set of contact members may occur subsequent to the selecting step.

A method of performing an orthopaedic procedure of the present disclosure may include determining a resection plane relative to an articular surface of a bone. The bone may extend along a longitudinal axis between a proximal end and a distal end. A periphery of the bone may establish a saddle extending axially between the proximal and distal ends. The method may include determining an orientation of at least one guide pin relative to the resection plane. The method may include determining positions of a first set of anatomical points along a non-articular surface of the bone. The first set of anatomical points may be distributed on opposite sides of the saddle. The method may include determining positions of a second set of anatomical points along the articular surface of the bone. The method may include configuring an alignment guide. The alignment guide may include a guide body coupled to at least one adjustment body along an interface. The guide body may include a guide passage. The guide body may carry a first set of adjustable contact members that may be distributed on opposite sides of a reference plane that bisects the guide body. The at least one adjustment body may carry a second set of adjustable contact members. The configuring step may include setting a position of each of the first and second sets of contact members based on the determined orientation. The configuring step may include setting the position of each of the first set of contact members based on the determined positions of the first set of anatomical points. The configuring step may include setting the position of each of the second set of contact members based on the determined positions of the second set of anatomical points. The method may include positioning the alignment guide relative to the bone according to the set positions. The method may include positioning the first set of contact members in contact with the first set of anatomical points such that the reference plane is substantially parallel to the longitudinal axis of the bone. The method may include positioning the second set of contact members in contact with the second set of anatomical points. The method may include inserting the at least one guide pin through the guide passage and into the bone subsequent to the positioning of the alignment guide.

In a further implementation, the method may include positioning a resection guide along the at least one guide pin such that an orientation of a resection slot of the resection guide may substantially correspond to the determined resection plane. The method may include moving a cutting instrument through the resection slot to resect a portion of the bone.

In a further implementation, the step of positioning the second set of contact members may occur subsequent to the step of positioning the first set of contact members.

In a further implementation, the bone may be a tibia. The step of determining the positions of the second set of anatomical points may include identifying the positions of the second set of anatomical points in a silhouette of the articular surface of the tibia. The second set of anatomical points may be distributed in a medial-lateral direction of the tibia.

In a further implementation, the second set of anatomical points may include a medial maximum and a tibial plafond of the articular surface of the tibia.

In a further implementation, the second set of anatomical points may include a tibial groove and a lateral maximum of the articular surface of the tibia.

In a further implementation, the interface may extend in a first direction that may be substantially perpendicular to the reference plane. The second set of adjustable contact members may be distributed in the first direction along the at least one adjustment body.

In a further implementation, the configuring step may include setting a position of the at least one adjustment body and the guide body relative to each other along the interface based on the determined positions of the first and second sets of anatomical points.

In a further implementation, the at least one adjustment body may include a set of adjustment bodies. The adjustment bodies may be interchangeably mountable to the guide body along the interface. The adjustment bodies may be dimensioned such that a distance between one or more adjacent pairs of the second set of contact members associated with the same anatomical points of the second set of anatomical points of the bone may differ within the set of adjustment bodies relative to the first direction. The step of configuring the alignment guide may include selecting the at least one adjustment body from the set of adjustment bodies based on the determined positions of the first and second sets of anatomical points. The step of setting the position of each of the second set of contact members may occur subsequent to the selecting step.

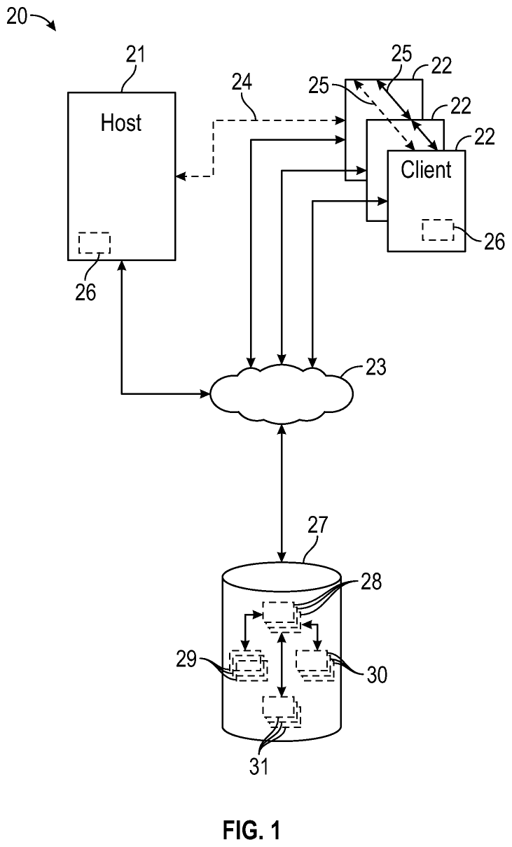

FIG. 1 illustrates an exemplary planning system 20 that may be utilized for planning various surgical procedures. The system 20 may be used for planning orthopaedic procedures, including pre-operatively, intra-operatively and/or post-operatively to create, edit, execute and/or review surgical plans for restoring functionality to joints.

The system 20 may include a host computer 21 and one or more client computers 22. The host computer 21 may be configured to execute one or more software programs. In some implementations, the host computer 21 is more than one computer jointly configured to process software instructions serially or in parallel.

The host computer 21 may be in communication with one or more networks such as a network 23 comprised of one or more computing devices. The network 23 may be a private local area network (LAN), a private wide area network (WAN), the Internet, or a mesh network, for example.

The host computer 21 and each client computer 22 may include one or more of a computer processor, memory, storage means, network device and input and/or output devices and/or interfaces. The input devices may include a keyboard, mouse, etc. The output device may include a monitor, speakers, printers, etc. The memory may, for example, include UVPROM, EEPROM, FLASH, RAM, ROM, DVD, CD, a hard drive, or other computer readable medium which may store data and/or other information relating to the planning techniques disclosed herein. The host computer 21 and each client computer 22 may be a desktop computer, laptop computer, smart phone, tablet, or any other computing device. The interface may facilitate communication with the other systems and/or components of the network 23.

Each client computer 22 may be configured to communicate with the host computer 21 directly via a direct client interface 24 or over the network 23. The client computers 22 may be configured to execute one or more software programs, such as a various surgical tools. The planning package may be configured to communicate with the host computer 21 either over the network 23 or directly through the direct client interface 24. In another implementation, the client computers 22 are configured to communicate with each other directly via a peer-to-peer interface 25.

Each client computer 22 may be operable to access and locally and/or remotely execute a planning environment 26. The planning environment 26 may be a standalone software package or may be incorporated into another surgical tool. The planning environment 26 may provide a display or visualization of one or more bone models and related images and one or more implant models via one or more graphical user interfaces (GUI). Each bone model, implant model, and related images and other information may be stored in one or more files or records according to a specified data structure.

The system 20 may include at least one storage system 27, which may be operable to store or otherwise provide data to other computing devices. The storage system 27 may be a storage area network device (SAN) configured to communicate with the host computer 21 and/or the client computers 22 over the network 23, for example. In implementations, the storage system 27 may be incorporated within or directly coupled to the host computer 21 and/or client computers 22. The storage system 27 may be configured to store one or more of computer software instructions, data, database files, configuration information, etc.

In some implementations, the system 20 is a client-server architecture configured to execute computer software on the host computer 21, which is accessible by the client computers 22 using either a thin client application or a web browser executing on the client computers 22. The host computer 21 may load the computer software instructions from local storage, or from the storage system 27, into memory and may execute the computer software using the one or more computer processors.

The system 20 may include one or more databases 28. The databases 28 may be stored at a central location, such as the storage system 27. In another implementation, one or more databases 28 may be stored at the host computer 21 and/or may be a distributed database provided by one or more of the client computers 22. Each database 28 may be a relational database configured to associate one or more bone models 29 and one or more implant models 30 to each other and/or a surgical plan 31. Each surgical plan 31 may be associated with a respective patient. Each bone model 29, implant model 30 and surgical plan 31 may be assigned a unique identifier or database entry. The database 28 may be configured to store data corresponding to the bone models 29, implant models 30 and surgical plans 31 in one or more database records or entries, and/or may be configured to link or otherwise associate one or more files corresponding to each respective bone model 29, implant model 30 and surgical plan 31. Bone models 29 stored in the database(s) 28 may correspond to respective patient anatomies from prior surgical cases, and may be arranged into one or more predefined categories such as sex, age, ethnicity, defect category, procedure type, etc.

Each bone model 29 may include information obtained from one or more medical devices or tools, such as a computerized tomography (CT), magnetic resonance imaging (MRI) machine and/or X-ray machine, that obtains one or more images of a patient. The bone model 29 may include one or more digital images and/or coordinate information relating to an anatomy of the patient obtained or derived from the medical device(s). Each implant model 30 may include coordinate information associated with a predefined design. The planning environment 26 may incorporate and/or interface with one or more modeling packages, such as a computer aided design (CAD) package, to render the models 29, 30 as two-dimensional (2D) and/or three-dimensional (3D) volumes or constructs.

The predefined design may correspond to one or more components. The implant models 30 may correspond to implants and components of various shapes and sizes. Each implant may include one or more components that may be situated at a surgical site including screws, anchors and/or grafts. Each implant model 30 may correspond to a single component or may include two or more components that may be configured to establish an assembly. Each bone model 29 and implant model 30 may correspond to 2D and/or 3D geometry, and may be utilized to utilized to generate a wireframe, mesh and/or solid construct in a display.

Each surgical plan 31 may be associated with one or more of the bone models 29 and implant models 30. The surgical plan 31 may include one or more revisions to a bone model 29 and information relating to a position of an implant model 30 relative to the original and/or revised bone model 29. The surgical plan 31 may include coordinate information relating to the revised bone model 29 and a relative position of the implant model 30 in predefined data structure(s). Revisions to each bone model 29 and surgical plan 31 may be stored in the database 28 automatically and/or in response to user interaction with the system 20.

One or more surgeons and other users may be provided with a planning environment 26 via the client computers 22 and may simultaneously access each bone model 29, implant model 30 and surgical plan 31 stored in the database(s) 28. Each user may interact with the planning environment 26 to create, view and/or modify various aspects of the surgical plan 31. Each client computer 22 may be configured to store local instances of the bone models 29, implant models 30 and/or surgical plans 31, which may be synchronized in real-time or periodically with the database(s) 28. The planning environment 26 may be a standalone software package executed on a client computer 22 or may be provided as one or more services executed on the host computer 21, for example.

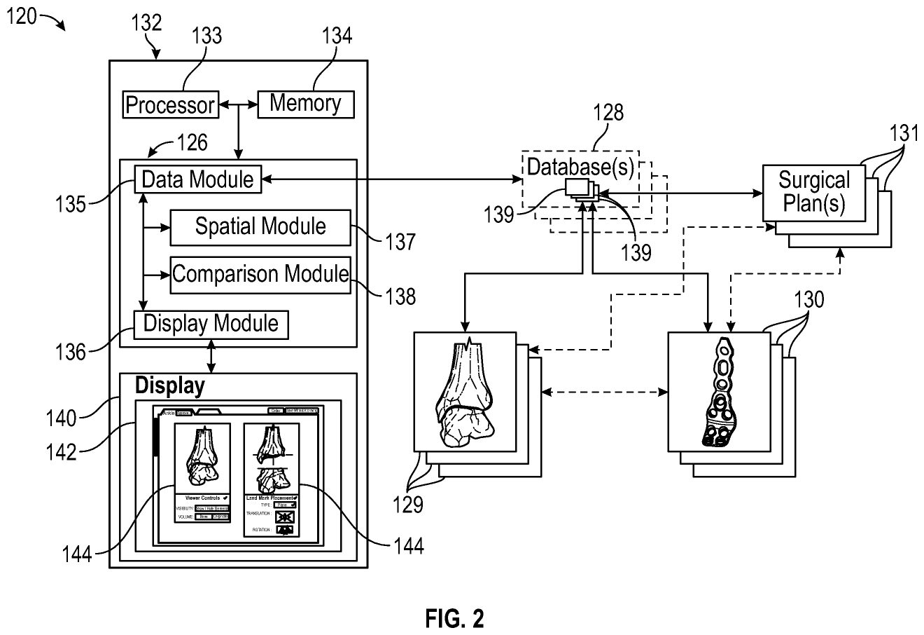

FIG. 2 illustrates an exemplary planning system 120 for planning a surgical procedure. In this disclosure, like reference numerals designate like elements where appropriate and reference numerals with the addition of one-hundred or multiples thereof designate modified elements that are understood to incorporate the same features and benefits of the corresponding original elements. The system 120 may be utilized to plan and implement various orthopaedic and other surgical procedures, such as an arthroplasty to repair a joint. The system 120 may be utilized in planning a resection of one or more bones that may be positioned for fusion. The system 120 may be utilized in planning placement of an implant, which may be utilized for fusion of resected bones such as a tibia and talus in an ankle repair. Although the planning system 120, instruments and methods disclosed herein primarily refer to repair of an ankle, it should be understood that the disclosed planning systems, instruments and methods may be utilized in the repair of other locations of the patient and other surgical procedures including repair of shoulder, wrist, hand, hip, knee and other joints, and repair of fractures.

The system 120 may include a computing device 132 including at least one processor 133 coupled to memory 134. The computing device 132 can include any of the computing devices disclosed herein, including the host computer 21 and/or client computer 22 of FIG. 1. The processor 133 may be configured to execute a planning environment 126 for creating, editing, executing and/or reviewing one or more surgical (e.g., pre-operative) plans 131 during pre-operative, intra-operative and/or post-operative phases of a surgery.

The planning environment 126 may include at least a data module 135, a display module 136, a spatial module 137 and a comparison module 138. Although four modules are shown, it should be understood that fewer or more than four modules may be utilized and/or one or more of the modules may be combined to provide the disclosed functionality.

The data module 135 may be configured to access, retrieve and/or store data and other information in the database(s) 128 corresponding to one or more bone model(s) 129, implant model(s) 130 and/or surgical plan(s) 131. The data and other information may be stored in one or more databases 128 as one or more records or entries 139. In some implementations, the data and other information may be stored in one or more files that are accessible by referencing one or more objects or memory locations references by the records or entries 139.

The memory 134 may be configured to access, load, edit and/or store instances of one or more bone models 129, implant models 130 and/or surgical plans 131 in response to one or more commands from the data module 135. The data module 135 may be configured to cause the memory 134 to store a local instance of the bone model(s) 129, implant model(s) 130 and/or surgical plan(s) 131 which may be synchronized with records 139 in the database(s) 128.

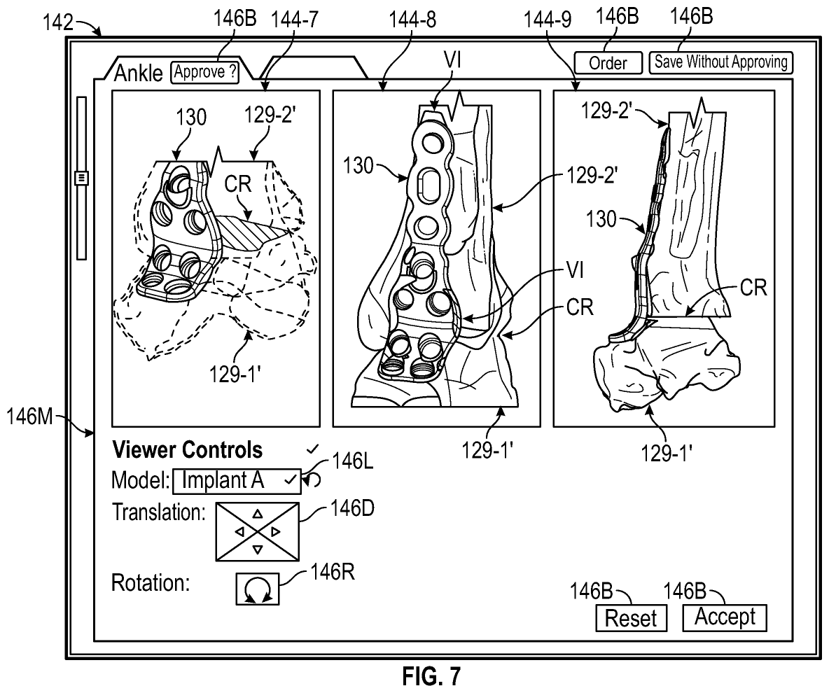

The implant model 130 may include one or more components. Exemplary implants may include bone plates configured to interconnect adjacent bones (see, e.g., FIG. 7) or bone fragments, base plates coupled to an articulation member, etc. The articulation member may have an articular surface dimensioned to mate with an articular surface of an opposed bone or implant.

The display module 136 may be configured to display data and other information relating to one or more surgical plans 131 in at least one graphical user interface (GUI) 142. The computing device 132 may be coupled to a display device 140. The display module 136 may be configured to cause the display device 140 to display information in the user interface 142. A surgeon or other user may interact with the user interface 142 via the planning environment 126 to create, edit, execute and/or review one or more surgical plans 131.

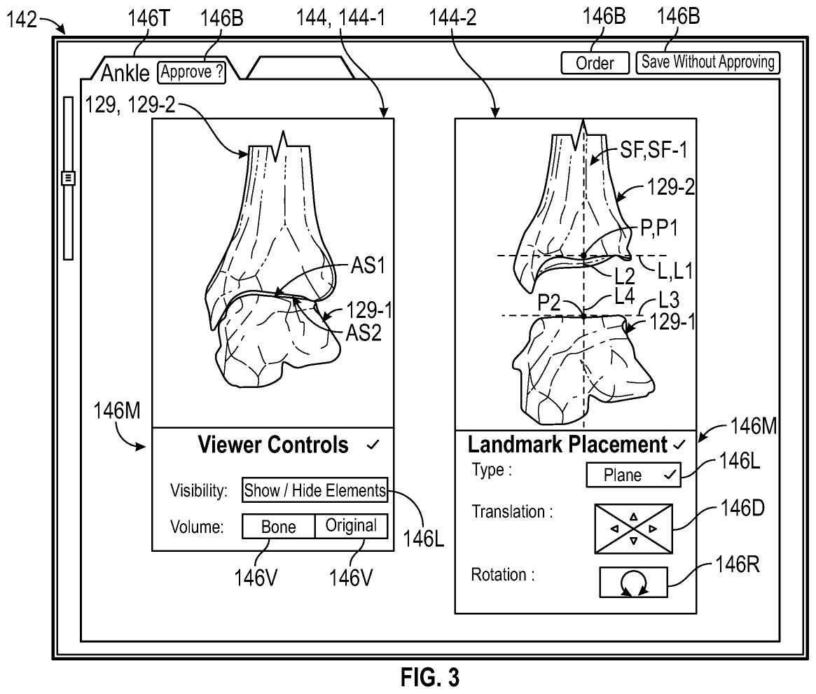

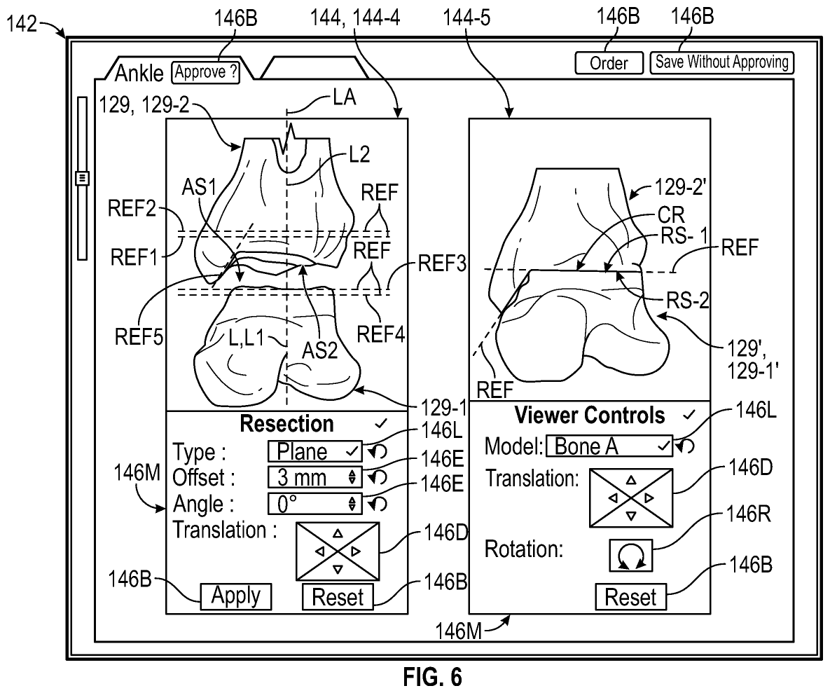

Referring to FIG. 3, with continuing reference to FIG. 2, the user interface 142 may include one or more display windows 144 and one or more objects 146. The objects 146 may include graphics such as menus, tabs and buttons accessible by user interaction, such as tabs 146T, buttons 146B, 146V, drop-down lists 146L, menus 146M, entry fields 146E (FIG. 6) and directional indicators 146D, 146R. Geometric objects, including selected bone model(s) 129, implant model(s) 130 (FIG. 7) and other information relating to the surgical plan 131, may be displayed in one or more of the display windows 144.

The display module 136 may be configured to display one or more selected bone models 129 and/or one or more selected implant models 130 (FIG. 7) in the display windows 144. The display module 136 may be configured such that the selected bone model 129 and/or selected implant model 130 may be selectively displayed and hidden (e.g., toggled) in one or more of the display windows 144 in response to user interaction with the user interface 142, which may provide the surgeon with enhanced flexibility in reviewing aspects of the surgical plan 131.

The data module 135 may be configured to access a first bone model 129-1 and a second bone model 129-1 from the database 128, which may occur automatically or in response to user interaction with the user interface 142. The data module 135 may be configured to store an instance of the first bone model 129-1 and second bone model 129-1 in the memory 134. The first bone model 129-1 and second bone model 129-2 may be associated with a joint. For example, one of the bone models 129 may be associated with a long bone such as a tibia, and another one of the bone models 129 may be associated with an adjacent bone such as a talus that cooperate to establish an ankle joint of a patient. In the implementation of FIG. 3, the second bone model 129-2 may be associated with a tibia, and the first bone model 129-1 may be associated with a talus. The display module 136 may be configured to display the first bone model 129-1 and second bone model 129-2 in at least one of the display windows 144 of the user interface 142.

The display windows 144 may include first and second display windows 144-1, 144-2. Although a particular number of display windows 144 are illustrated, it should be understood that the user interface 142 may be configured with any number of display windows 144 in accordance with the teachings disclosed herein. The display windows 144-1, 144-2 may be configured to display a two-dimensional (2D) and/or three-dimensional (3D) representation of the selected bone models 129.

The first display window 144-1 may be configured to display the first bone model 129-1 and second bone model 129-2 relative to each other. The spatial module 137 may be configured to position the bone models 129-1, 129-2 in contact with each other at a specified or defined position and orientation, which may be according to user interaction with the window 144-1, menu 146M, and/or other objects 146 of the user interface 142.

The surgeon or assistant may interact with the display window 144-1 or another portion of the user interface 142 to move the selected bone model 129 and/or selected implant model 130 (FIG. 7) in 2D space (e.g., up, down, left, right) and/or 3D space (e.g., rotation, tilt, zoom, etc.), which may occur in response to interaction with the directional indicators 146D, 146R.

The second display window 144-2 may be configured to display the first bone model 129-1 and second bone model 129-2 in spaced relationship relative to each other. The surgeon or assistant may interact with the second display window 144-2 or another portion of the user interface 142 to associate one or more landmarks L with the selected bone models 129. The landmarks L may include one or more anatomical (e.g., reference) points P along the anatomy (e.g., P1-P2), one or more planes (e.g., L1-L4) and one or more surface features SF. Exemplary landmarks include a longitudinal (e.g., tibial) axis, saddle, sagittal plane, coronal plane and transverse plane.

Exemplary surface features SF can include a saddle SF-1 established by a periphery of the bone. The saddle SF-1 can be a ridge or crest along the periphery of the bone that extends a distance between proximal and distal ends of the bone relative to a longitudinal axis of the bone.

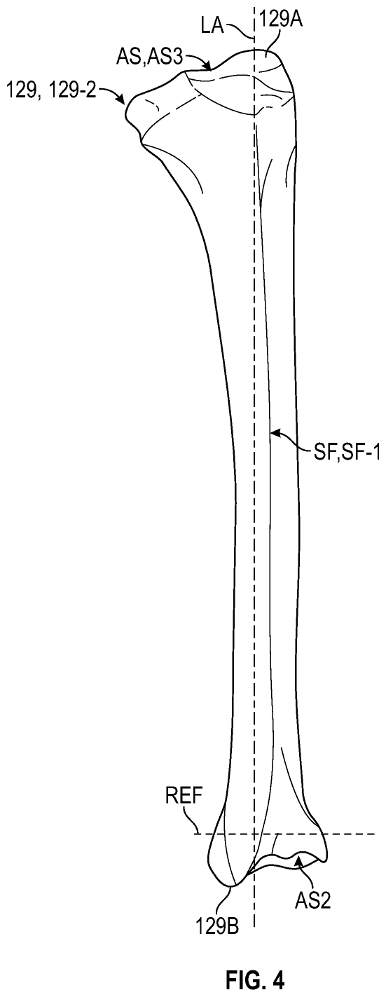

In the implementation of FIG. 4, the bone model 129-2 may be associated with a long bone, such as a tibia. The bone model 129-2 can extend along a longitudinal axis LA between a first (e.g., proximal) end 129A and a second (e.g., distal) end 129B. The second end 129B may be associated with an articular surface AS2. The first end 129A may be associated with an articular surface AS3. Each of the articular surfaces AS2, AS3 may be associated with a respective joint. For example, the articular surface AS2 may cooperate with an articular surface AS1 of the first bone model 129-1 (FIG. 3) to establish an ankle joint. The articular surface AS3 may cooperate with an articular surface of a pelvis to establish a hip joint. A reference plane REF may be established relative to the saddle SF-1 and/or longitudinal axis LA. The reference plane REF may be substantially perpendicular or otherwise transverse to the saddle SF-1 and/or longitudinal axis LA. For the purposes of this disclosure, the terms “substantially,” “about” and “approximately” mean±5 percent of the stated value or relationship unless otherwise indicated. The reference plane REF may be associated with a resection plane and may be established utilizing any of the techniques disclosed herein.

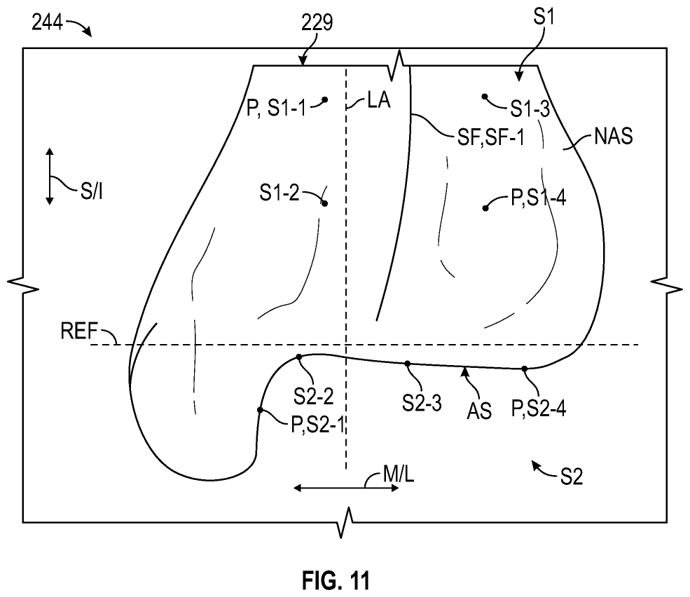

In the implementation of FIG. 11, bone model 229 is associated with a saddle SF-1 and one or more anatomical points P. The bone model 229 may be displayed in a display window 244. The anatomical points P can include a first set S1 of anatomical points P and a second set S2 of anatomical points P. Each of the first and second sets S1, S2 can include one or more anatomical points P, as illustrated by points S1-1 to S1-4 and points S2-1 to S2-4, respectively. The first set S1 of anatomical points S1-1 to S1-4 may be established along a non-articular surface NAS of a bone associated with the bone model 229. The first set S1 of anatomical points S1-1 to S1-4 may include one or more anatomical points S1-1 to S1-4 distributed axially relative to a longitudinal axis LA of the bone model 229. The first set S1 of anatomical points P may be spaced apart at respective distances from a reference plane REF, which may be associated with a resection plane. The first set S1 of anatomical points P may include one or more anatomical points S1-1 to S1-4 distributed on opposite sides of the saddle SF-1, such as adjacent points S1-1, S1-2 and adjacent points S1-3, S1-4. The second set S2 of anatomical points S2-1 to S2-4 may be established along an articular surface AS2 of the bone model 229. The points S2-1 to S2-4 can be distributed in a medial/lateral direction M/L of the patient.

The bone model 229 may be associated with a long bone, such as a tibia. The anatomical points S2-1 to S2-4 may substantially correspond to respective positions of a medial maximum, tibial groove, tibial plafond and lateral maximum along the articular surface AS of the tibia, such as a distal tibia. The anatomical points S2-1 to S2-4 may be spaced apart at respective distances from the reference plane REF. In other implementations, the anatomical points S2-1 to S2-4 may substantially correspond to respective positions along the articular surface of the proximal tibia.

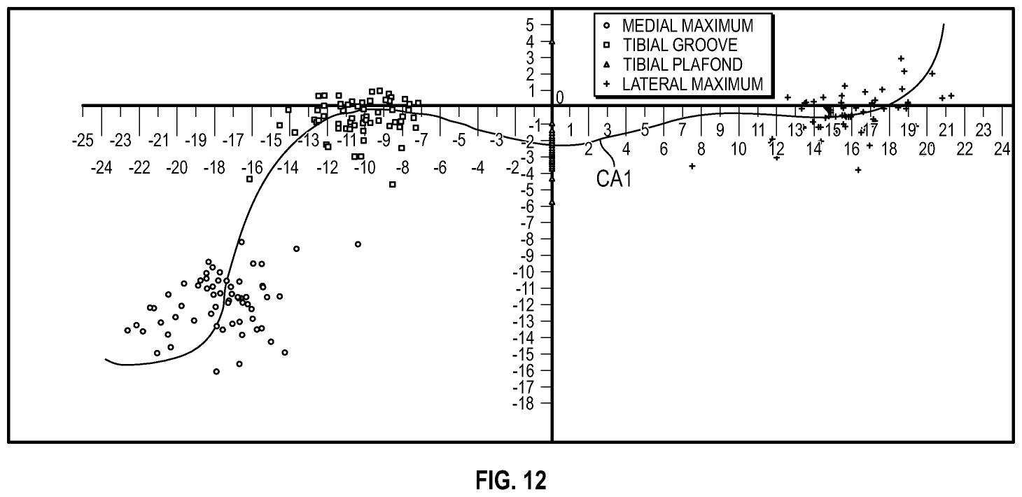

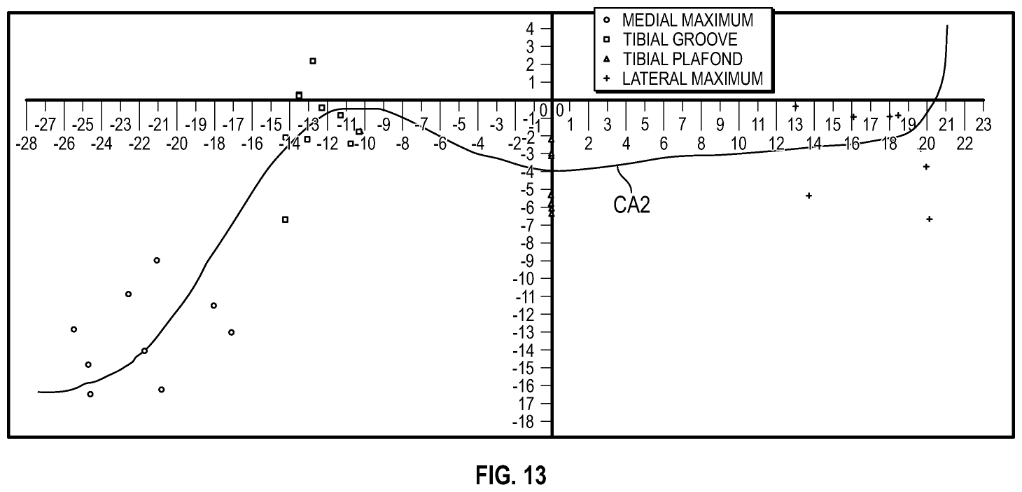

Referring back to FIGS. 2-3, the spatial module 137 may be configured to determine one or more landmarks L based on evaluating a profile of the selected bone model 129, including any of the landmarks and surface features disclosed herein. The profile of the selected bone model 129 can be compared to one or more profiles of representative bones in the database 128. Applicants have determined through anatomical and pathological analysis that the relative positions of the medial maximum, tibial groove, tibial plafond and lateral maximum along the articular surface of the distal tibia of patients can be highly correlated. Determining these positions can be highly reproduceable for various anatomies including different shapes, sizes and spacings that may be encountered by the surgeon. FIG. 12 illustrates a plot of data points associated with a total of 61 anatomical samples of a silhouette of the articular surface of a bone of the anatomy, such as the distal tibia, normalized to the tibial plafond and an associated best fit curve CA1. FIG. 13 illustrates a total of 9 pathological samples of a silhouette of the articular surface of a bone of the anatomy, such as the distal tibia, normalized to the tibial plafond and an associated best fit curve CA2. Each of the curves CA1, CA2 can be expressed as a polynomial and can closely approximate a profile of the articular surface of the distal tibia.

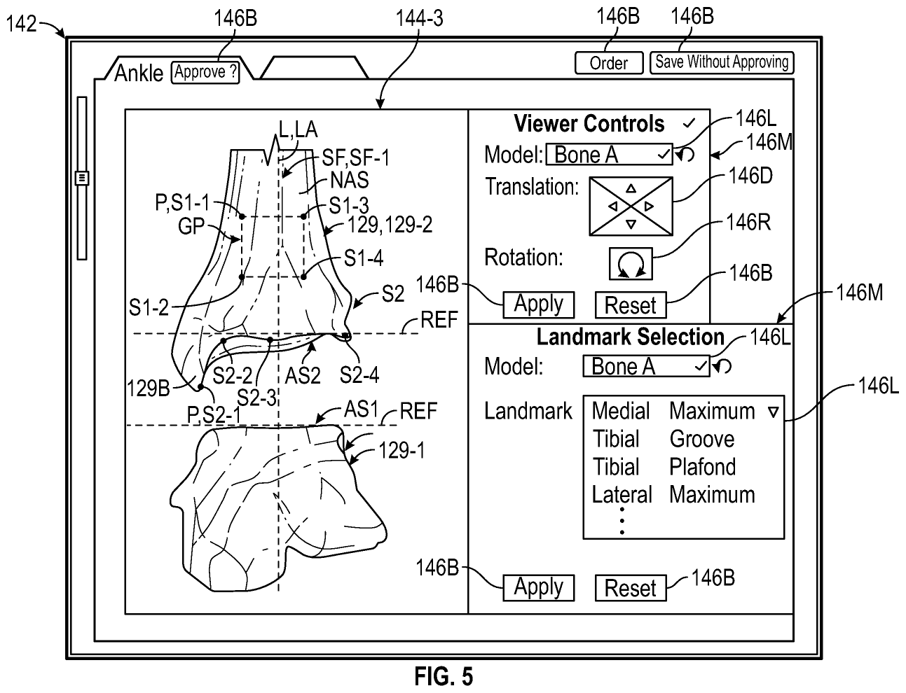

Referring to FIG. 5, with continuing reference to FIGS. 2-3, the user interface 142 may include a third display window 144-3. The third display window 144-3 may be configured to display a silhouette (e.g., side view) of the selected bone model 129, such as the bone model 129-2, and an adjacent bone model 129 associated with the same joint, such as the bone model 129-1.

The surgeon or assistant may interact with the display window 144-3 or another portion of the user interface 142 to specify one or more aspects of, or modifications to, the surgical plan 131. The aspects may include one or more anatomical points P of the respective bone model 129, such as the second set S2 of anatomical points P (see also FIG. 11). In implementations, the surgeon or other user may interact with a drop down list 146L of the menu 146M to select and specify one or more anatomical points P along the bone model 129, such as the medial maximum, tibia groove, tibial plafond and/or lateral maximum of the distal tibia. The surgeon or other user may interact with the display window 144-3 to set the position of each respective anatomical point P, such as the anatomical points S2-1 to S2-4 associated with medial maximum, tibia groove, tibial plafond and/or lateral maximum of the tibia. In implementations, the spatial module 137 may be configured to determine and set the position of each landmark L selected from the list 146L in response to selection of the respective bone model 129 from another drop down list 146L or another portion of the menu 146M. The spatial module 137 may be configured to determine the position of the selected landmark L based on evaluating a profile of the selected bone model 129. The spatial module 137 may be configured to compare the best fit curves CA1 and/or CA2 (FIGS. 12-13) to the profile of the selected bone model 129 to determine the position of the selected landmark(s) L. The surgeon or other user may interact with the user interface 142 to modify the position of each of the landmarks L established by the spatial module 137, including the anatomical points P.

Referring to FIG. 6, with continuing reference to FIGS. 2-3, the user interface 142 may include fourth and fifth display windows 144-4, 144-5. The surgeon or assistant may interact with the display windows 144-4, 144-5 or another portion of the user interface 142 to specify one or more aspects of, or modifications to, the surgical plan 131. The modifications may include one or more resection planes REF associated with a respective one of the bone models 129 (see also FIG. 5). The fourth display window 144-4 may be configured to display each reference plane REF relative to the respective bone model 129. The bone models 129-1, 129-2 may include respective articular surfaces AS1, AS2 associated with a joint, such as an ankle joint. Each reference plane REF may be associated with a respective depth inward from the respective articular surface AS1/AS2 of the bone model 129 (e.g., 1 mm, 2 mm, etc.). The reference planes REF may be substantially perpendicular to a landmark L of the respective bone model 129, such as the landmarks L1, L2. The reference planes REF can include reference planes REF3 and/or REF4 oriented relative to the landmark L1. The reference planes REF can include reference planes REF1 and/or REF3 oriented relative to the landmark L2. The landmark L1 may extend through the bone model 129-1 and may be substantially aligned with the longitudinal axis LA of the bone model 129-2. The landmark L2 may be associated with the longitudinal axis LA of the bone model 129-2 (see also FIG. 4). One or more of the reference planes REF may be transverse to and may intersect another reference plane REF (e.g., reference plane REF5 relative to reference planes REF1-REF2).

The spatial module 137 may be configured to set a position of a first reference plane REF (e.g., REF3 or REF4) with respect to the first bone model 129-1 in response to user interaction with the user interface 142. The spatial module 137 may be configured to set a position of a second reference plane REF (e.g., REF1 or REF2) with respect to the second bone model 129-2 in response to user interaction with the user interface 142. For example, the surgeon or assistant may adjust a position (e.g., depth) and/or angle of a selected reference plane REF in response to user interaction with the user interface 142. The surgeon or assistant may interact with one of the entry fields 146E to specify an offset or depth associated with the reference plane REF relative to the articular surface AS1/AS2.

The spatial module 137 may be configured to generate a first iteration of respective first and second (e.g., resected) bone models 129-1′, 129-2′. The bone models 129-1′, 129-2′ may exclude volumes of the bone models 129-1, 129-2 between the respective reference planes REF and articular surfaces AS1, AS2 of the bone models 129-1, 129-2, as illustrated in the fifth display window 144-5. The bone models 129-1′, 129-2′ may be separate models or may be stored as one or more revisions to the respective bone models 129-1, 129-2.

The spatial module 137 may be configured to establish a first resection (e.g., contact) surface S1 of the first bone model 129-1′ according to a first reference plane REF (e.g., REF1 or REF2) and may be configured to establish a second resection (e.g., contact) surface S2 of the second bone model 129-2′ along a second reference plane REF (e.g., REF3 or REF4), as illustrated in the fifth display window 144-5. The first and second resection surfaces RS-1, RS-2 of the bone models 129-1′, 129-2′ may be opposed relative to each other and may be positioned apart and/or in abutment in the display windows 144. The fifth display window 144-5 may be dynamically linked to the fourth display window 144-4 such that selections and/or adjustments to the respective reference plane REF associated with the fourth display window 144-4 cause a position of the respective resection surface RS-1/RS-2 to change in the fifth display window 144-5. The display module 136 may be configured to display the iteration of the bone models 129-1′, 129-2′ in the fifth display window 144-5 in response to adjusting or otherwise setting the resection planes REF. The surgeon or assistant may interact with the user interface 142 to obtain a visualization of resection depths, which may be adjusted prior to approval of the surgical plan 131.

The spatial module 137 may be configured to position the first resection surface RS-1 associated with the first bone model 129-1′ in contact with the second resection surface RS-2 of associated with the second bone model 129-2′ along a contact region CR. The contact region CR may be a region of bone-to-bone contact between the resection surfaces RS-1, RS-2. The contact region CR may be continuous or may be discontinuous including two or more localized regions of contact. The localized regions of contact may be separated by a space due to surface depression(s) or other contouring along the resection surfaces RS-1, RS-2.

The surgeon or assistant may interact with the directional indicators 146D, 146R or another portion of the user interface 142 to adjust or otherwise set a relative position between the bone models 129-1′, 129-2′ along the contact region CR. The surgeon or assistant may interact with the user interface 142 to evaluate aspects of the bone models 129-1′, 129-2′ relative to the contact region CR and associated reference planes REF.

Referring to FIG. 7, with continuing reference to FIGS. 2-3 and 6, the display module 136 may be configured to display one or more implant models 130 in one or more of the display window(s) 144 of the user interface 142. Each implant model 130 can be associated with any of the implants disclosed herein. The display windows 144 may include seventh, eighth and ninth display windows 144-7, 144-8, 144-9. The spatial module 137 may be configured to position the selected implant model(s) 130 relative to the first and second bone models 129-1′, 129-2′ automatically and/or in response to user interaction with the user interface 142.

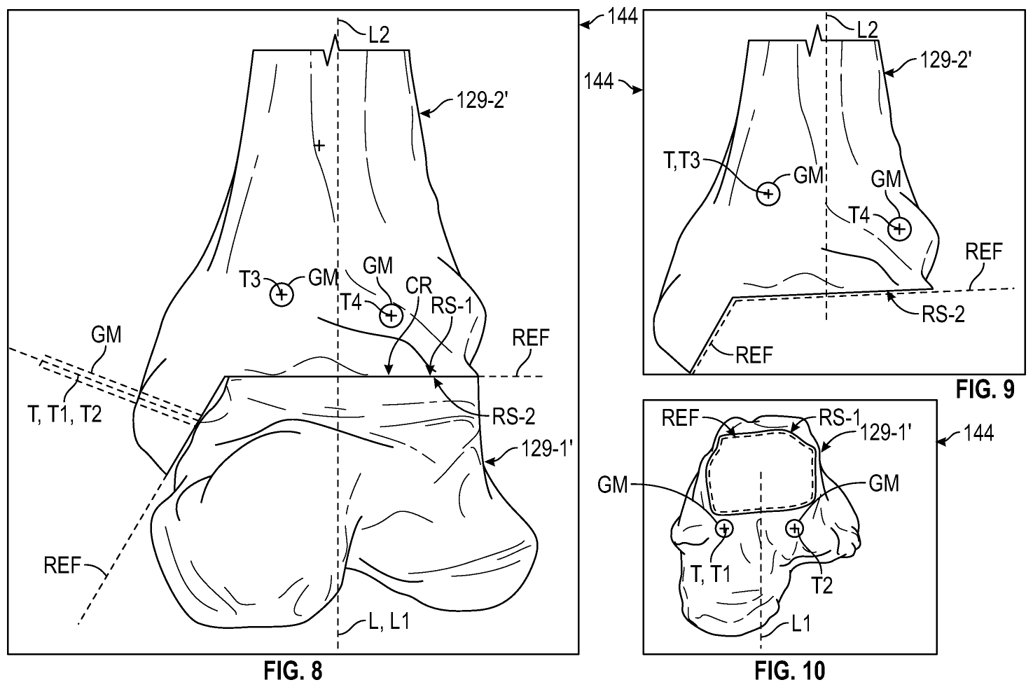

Referring to FIGS. 8-10, with continuing reference to FIGS. 2-3, the spatial module 137 may be configured to determine one or more trajectories T for placement of respective guide members GM. Exemplary guide members may include guide pins (e.g., Kirschner wires), fasteners, etc. The guide members GM can be configured for insertion into bone to guide placement of surgical instrument(s). The trajectories T may include first through fourth trajectories T1-T4. Each trajectory T may include a location of insertion of the guide member GM along a surface of the bone model 129-1′/129-2′ and/or orientation along an axis passing through the location. The trajectories T may be associated with respective guide members GM. The spatial module 137 may be configured to determine the trajectories T based on one or more of the landmarks, including any of the landmarks disclosed herein.

The trajectories T1-T4 may be associated with respective positions along the bone models 129-1′, 129-2′ relative to the contact region CR (FIG. 8). The spatial module 137 may be configured to establish the trajectories T1-T4 based on one or more landmarks L, such as the landmarks L1, L2, surface features SF, and/or first and second sets S1, S2 of the anatomical points P including any or all of the points S2-1 to S2-4 (FIG. 5), the defined reference planes REF (see also FIG. 5), and/or a geometry of an instrument selected to position the respective guide member(s) GM. The trajectories T1-T4 may be established relative to predefined offsets relative to the respective reference planes REF based on the dimensions of one or more surgical instruments specified in the surgical plan 131 for placement of the guide members GM, including any of the alignment guides and other surgical instruments disclosed herein. The surgeon or assistant may interact with the user interface 142 to adjust a position and/or orientation of each trajectory T. In implementations, the surgeon may interact with the user interface 142 to approve the trajectories T and the defined reference planes REF associated with the resection surfaces RS-1, RS-2.

The comparison module 138 may be configured to generate one or more settings or dimensions associated with an instrument based on the trajectories T. Exemplary instruments may include cutting blocks, alignment guides, etc., including any of the instruments disclosed herein. The dimensions may be utilized to fabricate a patient-specific instrument for implementation of the surgical plan 131. The trajectories T, settings and/or dimensions may be stored in the surgical plan 131. The surgeon may interact with the user interface 142 to approve the surgical plan 131, which may be stored in the database 128 for later retrieval.

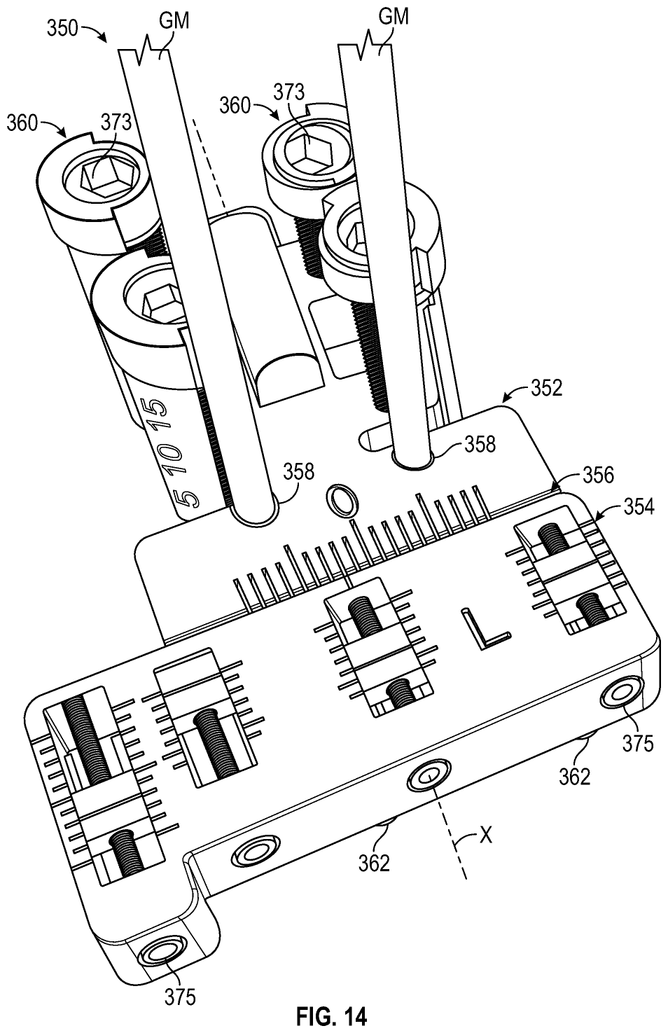

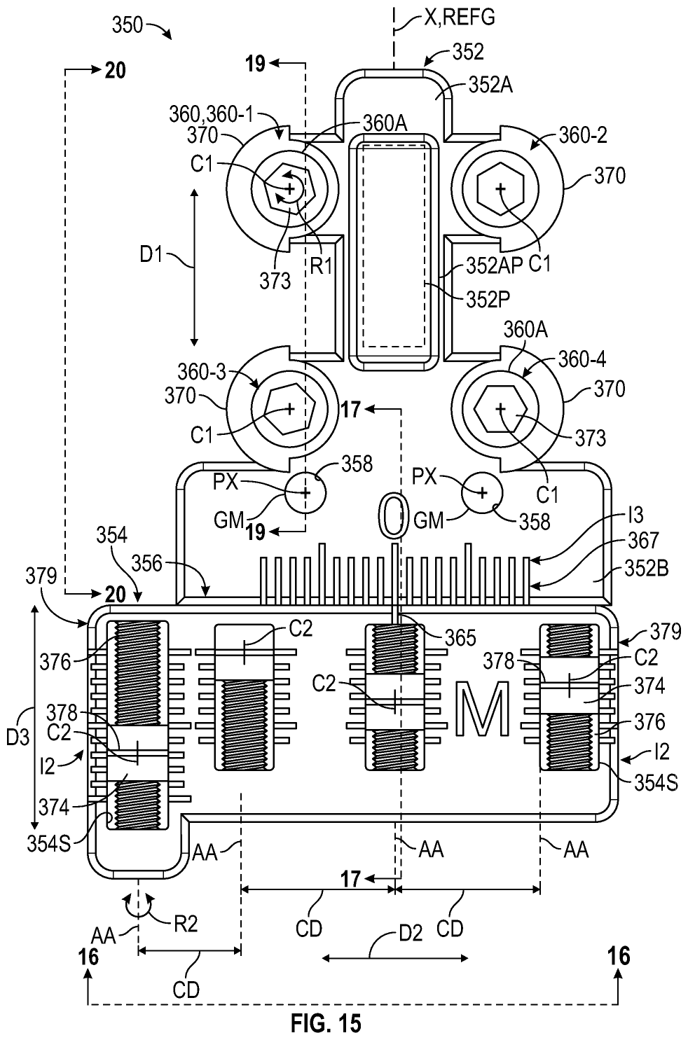

FIGS. 14 and 15 illustrate an exemplary alignment guide 350 that may be utilized for various surgical procedures, including preparation of a surgical site. The alignment guide 350 may be configured to position one or more guide members GM relative to bone or other tissue based on one or more predetermined trajectories. The alignment guide 350 may be utilized in an orthopaedic procedure such as an ankle reconstruction to facilitate the removal of bone along an articular surface of a tibia and/or talus. The alignment guide 350 may be utilized to repair other joints of the body, including hip, shoulder and knee joints, and other bones of the body. A location of the bone to be removed may be determined intraoperatively or during preoperative planning utilizing the system 120.

Referring to FIG. 15, with continuing reference to FIG. 14, the alignment guide 350 can include a guide (e.g., first) body 352 and at least one adjustment (e.g., second) body 354 secured to the guide body 352. The guide body 352 and adjustment body 354 may be integrally formed or may be separate and distinct components. The adjustment body 354 may be releasably secured or otherwise coupled to the guide body 352 along an interface 356. The guide body 352 may extend along a guide (e.g., longitudinal) axis X between a first (e.g., proximal) end portion 352A and a second (e.g., distal) end portion 352B. The guide body 352 may be dimensioned to extend along a reference plane REFG. The reference plane REFG may bisect the guide body 352 and may extend along the guide axis X. Opposite sides of the guide body 352 may be substantially symmetrical relative to the reference plane REFG. The interface 356 may be substantially perpendicular or otherwise transverse to the guide axis X and/or reference plane REFG.

The alignment guide 350 may be utilized in the positioning of one or more guide members GM (FIG. 14) relative to each other and relative bone or other tissue at a surgical site. The guide members GM may include any of the guide members disclosed herein, such as a guide pin. The guide body 352 can include at least one or more guide passages 358. Each of the guide passages 358 can be dimensioned to receive a respective guide member GM. The guide body 352 can guide passages 358 distributed on opposite sides of the reference plane REFG. Each guide passage 358 can be dimensioned to set a trajectory of the guide member GM relative to bone or other tissue for orienting another surgical instrument, such as a drill or cutting guide for removing a portion of bone or other tissue. Although two guide passages 358 are shown, the alignment guide 350 can include fewer or more than two guide passages 358 such as only one guide passage 358.

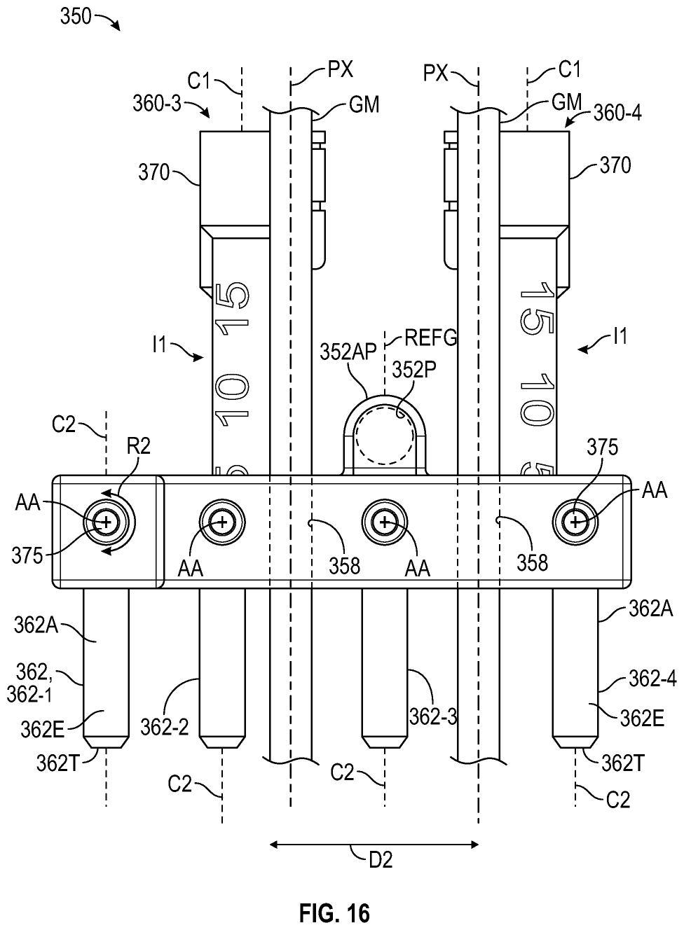

Referring to FIGS. 15 and 16, with continuing reference to FIG. 14, the guide passage 358 can extend along a respective passage axis PX. The passage axis PX may be substantially perpendicular or otherwise transverse to the guide axis X. The passage axis PX can establish a trajectory of a respective guide member GM received in and through the guide passage 358. The guide member GM may be positioned at a predetermined trajectory relative to bone or other tissue along a surgical site. The trajectory may be determined intraoperatively or during preoperative planning utilizing the system 120 and may be specified in a surgical plan 131 (FIG. 2).

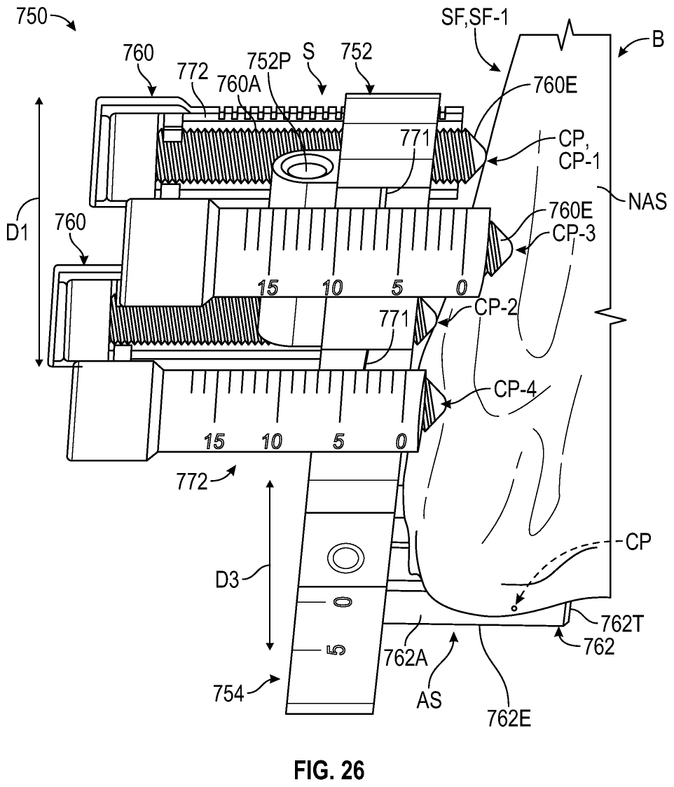

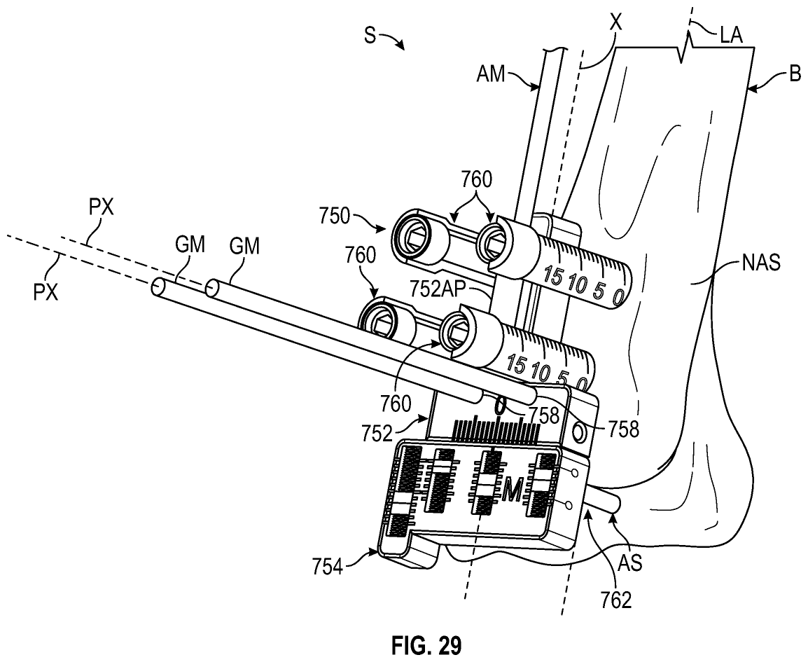

The guide body 352 can include an alignment portion 352AP for orienting the alignment guide 350. The alignment portion 352AP can establish a passage 352P (shown in dashed lines, see also FIG. 26). The passage 352P may be dimensioned to extend along the reference plane REFG. The passage 352P may be substantially parallel to the guide axis X. The passage 352P can be dimensioned to receive an alignment member AM (see, e.g., FIG. 29). The alignment member AM may include an elongated rod. The alignment member AM may comprise a radiopaque material and may be utilized intraoperatively with radiographic imagery and/or through direct visualization to position the guide body 352 relative to a landmark, such as a longitudinal axis of the respective bone (see, e.g., axis LA of FIG. 29, see also FIG. 4).

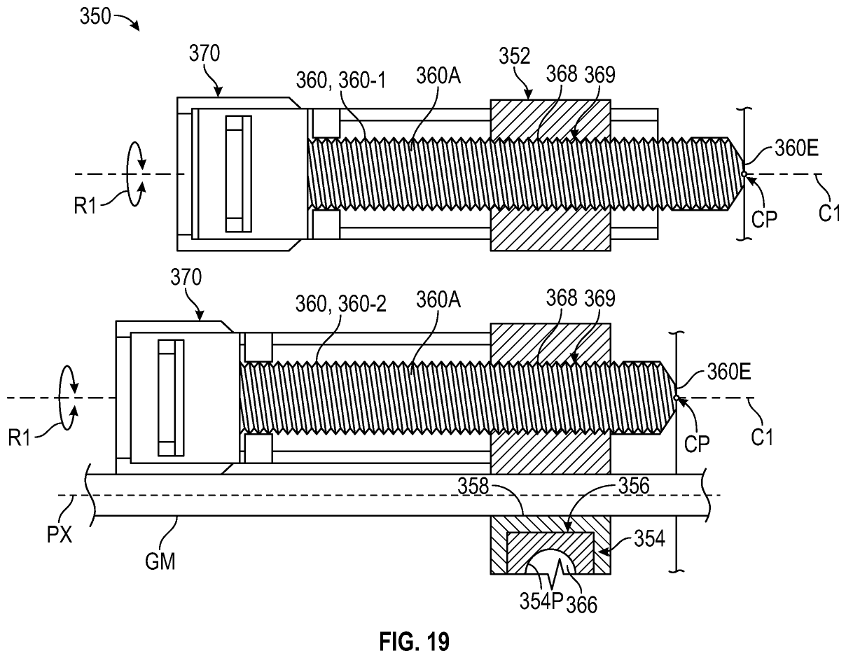

The guide body 352 and adjustment body 354 may be configured to set a trajectory of one or more guide members GM relative to bone or other tissue. The guide body 352 may carry a first set of adjustable contact members 360. Each of the contact members 360 can be dimensioned to contact bone or other tissue at respective contact points CP (see, e.g., FIG. 19). The adjustment body 354 may carry a second set of adjustable contact members 362 (FIG. 16). Each of the contact members 362 can be dimensioned to contact bone or other tissue at respective contact points CP. The contact members 360, 362 may be configured or set according to respective settings to establish an orientation of the alignment guide 350 relative to bone at a surgical site and establish a trajectory of each guide member GM insertable through the guide passage 358 relative to the bone or other tissue. The settings can be specified in a surgical plan 131 (FIG. 2).

The alignment guide 350 can include a total of four contact members 360 (indicated at 360-1 to 360-4) and a total of four contact members 362 (indicated at 362-1 to 362-4). The alignment guide 350 can include fewer or more than four contact members 360 and four contact members 362, such as only one or two contact members 360 and/or only one or two contact members 362. The first set of contact members 360 may include one or more contact members 360 distributed on opposite sides of the reference plane REFG of the guide body 352.

The positions of the first set of contact members 360 relative to the guide body 352 and the positions of the second set of contact members 362 relative to the adjustment body 354 may be determined intraoperatively or during preoperative planning utilizing the system 120. Each of the contact members 360, 362 may be independently adjustable and set according to a predetermined position, which may be specified in a surgical plan 131 (FIG. 2). In other implementations, two or more of the contact members 360, 362 are simultaneously moveable to respective positions.

The first set of contact members 360 can be distributed in a first direction D1 and/or second direction D2 along the guide body 352. The second set of contact members 362 can be distributed in the second direction D2 along the adjustment body 354. The first and second directions D1, D2 can be established relative to the guide axis X. The first direction D1 can be substantially parallel to the guide axis X, and the second direction D2 can be substantially perpendicular to the guide axis X, or vice versa. The interface 356 may be dimensioned to extend in the second direction D2. The direction D2 may be substantially perpendicular or otherwise transverse to the reference plane REFG of the guide body 352.

The guide body 352 and adjustment body 354 may be moveable relative to each other along the interface 356 to adjust a position of the first set of contact members 360 and the second set of contact members 362 relative to each other. The position of the guide body 352 and adjustment body 354 relative to each other may be determined intraoperatively or during preoperative planning utilizing the system 120 and can be specified in a surgical plan 131 (FIG. 2).

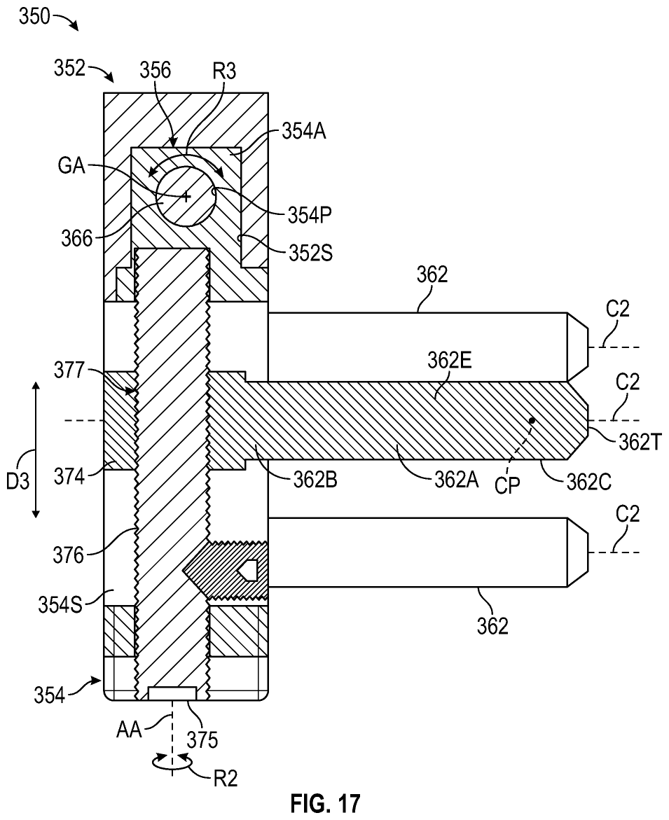

Various techniques may be utilized to secure the guide body 352 and adjustment body 354 to each other. Referring to FIG. 17, with continuing reference to FIGS. 15-16, a portion 354A of the adjustment body 354 may be received in an elongated slot 352S established in the guide body 352, although an opposite arrangement may be utilized. The portion 354A of the adjustment body 354 may include a passage 354P dimensioned receive a guide shaft 366. The adjustment body 354 may be carried on the guide shaft 366. The adjustment body 354 can cooperate with the guide shaft 366 to establish a worm drive mechanism. Ends of the guide shaft 366 may be supported by the guide body 352. The guide shaft 366 can be a threaded shaft that extends along a guide axis GA. The passage 354P may include threading that meshes with the threading along the guide shaft 366. The adjustment body 354 may be translatable along the guide axis GA in response to rotation of the guide shaft 366 in a direction R3 about the guide axis GA (see also FIG. 20). Tooling (e.g., Allen wrench) can be utilized to engage the guide shaft 366 at an interface 381 (FIG. 20) to rotate or otherwise move the guide shaft 366. Translation of the adjustment body 354 along the guide shaft 366 can cause the adjustment body 354 to move in the direction D2 (FIG. 15) relative to the guide body 352. The guide shaft 366 may be removeable from the passage 354P to unmount or otherwise uncouple the guide body 352 and adjustment body 354 from each other.

Referring back to FIG. 15, a relative position between the guide body 352 and adjustment body 354 may be configured relative to indicia I3. The indicia I3 may be established adjacent to the interface 356. The indicia I3 may be associated with respective positions of the adjustment body 354 relative to the guide body 352 in the direction D2. The surgeon or assistant may move the adjustment body 354 relative to the indicia I3 according to one or more settings specified in a surgical plan 131 generated by the system 120 (FIG. 2).

In implementations, the indicia I3 are established by an indicator 365 relative to a ruler 367. The adjustment body 354 can be dimensioned to set a position of the indicator 365 relative to the ruler 367 in response to relative movement between the guide body 352 and adjustment body 354. The indicator 365 may be established along a face of the adjustment body 354, and the ruler 367 may be established along a portion of the guide body 352, although the opposite arrangement may be utilized. The indicator 365 may be aligned with a selected position along the ruler 367 to indicate a position of the second set of contact members 362 relative to the guide body 352. Relative movement between the guide body 352 and adjustment body 354 along the interface 356 can provide an extra degree of control and precision in positioning the alignment guide 350 relative to the anatomy.

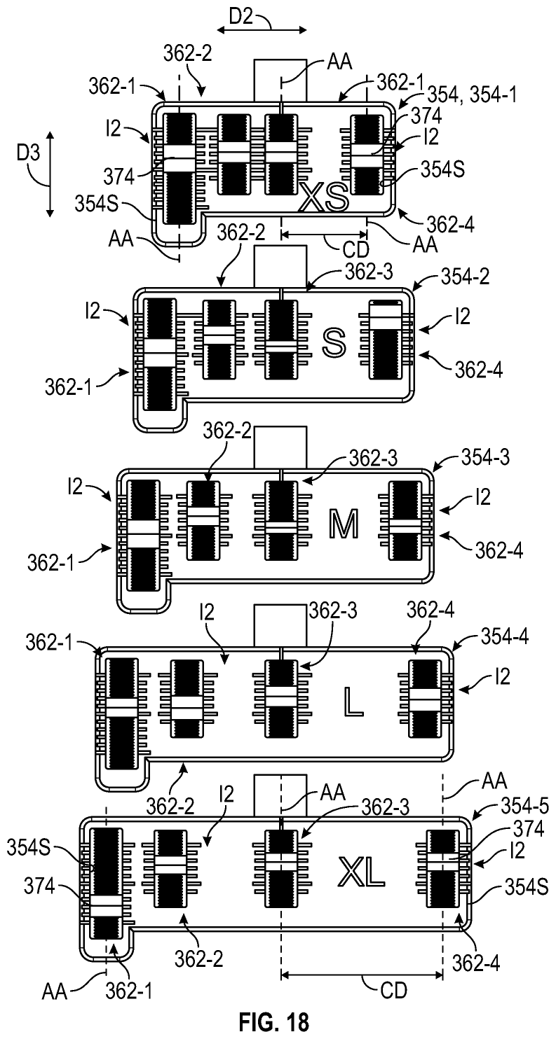

The alignment guide 350 can include a set of adjustment bodies 354, as illustrated by adjustment bodies 354-1 to 354-5 in FIG. 18. The adjustment bodies 354-1 to 354-5 can be provided with other portions of the alignment guide 350 as a kit to the surgeon. The adjustment bodies 354-1 to 354-5 may be interchangeably mountable to the guide body 352 along the interface 356. The adjustment bodies 354-1 to 354-5 can be dimensioned to have various shapes and sizes, such as extra-small (XS), small(S), medium (M), large (L) and/or extra-large (XL), to accommodate different anatomies and surface profiles. Although a total of five adjustment bodies 354 are illustrated in FIG. 18, it should be understood that fewer or more than five adjustment bodies 354 may be utilized with the alignment guide 350, such as only one or two adjustment bodies 354. Each of the adjustment bodies 354-1 to 354-5 can be configured to carry a respective second set of the adjustable contact members 362. A total number of the contact members 362 in the second set of contact members 362 can be equal for every one of the adjustment bodies 354-1 to 354-5 in the set of adjustment bodies 354. In implementations, each of the adjustment bodies 354-1 to 354-5 can have a total of four contact members 362. The second set of contact members 362 of each adjustment body 354-1 to 354-5 in the set of the adjustment bodies 354 can be associated with, and can be dimensioned to establish contact with, the same set of anatomical points of a bone of the anatomy, such as the distal tibia.

Referring to FIGS. 16-17 and 19-20, with continuing reference to FIG. 15, each of the contact members 360, 362 can include an elongated main body 360A, 362A and a respective engagement portion 360E, 362E. The contact members 360, 362 can have various geometries and configurations to establish a trajectory of the passage axes PX (FIG. 15) and associated guide member(s) GM relative to a surgical site. In implementations, the contact members 360, 362 are elongated pins. The elongated bodies 360A, 362A can extend along respective contact axes C1, C2 to the respective engagement portions 360E, 362E. The engagement portions 360E, 362E can establish free ends 360T, 362T of the contact members 360, 362 or can be spaced apart from free ends 360T, 362T of the contact members 360, 362.

The contact members 360, 362 can be dimensioned to establish contact with bone or other tissue along respective contact points. The first set of contact members 360 can be dimensioned such that a contact point CP is established by the engagement portion 360E at the free end 360T of the respective contact member 360, as illustrated by FIGS. 17 and 19 (see also FIG. 26). The contact members 362 can be dimensioned such that respective contact points CP are established by the engagement portion 362E along a periphery of the respective contact member 362. The second set of contact members 362 can be dimensioned such that respective contact points CP are spaced apart from the free ends 362T of the respective contact member 362 (see, e.g., FIGS. 26-28). In other implementations, the contact members 362 can be dimensioned such that respective contact points CP are established at the free ends 362T of the contact members 362.

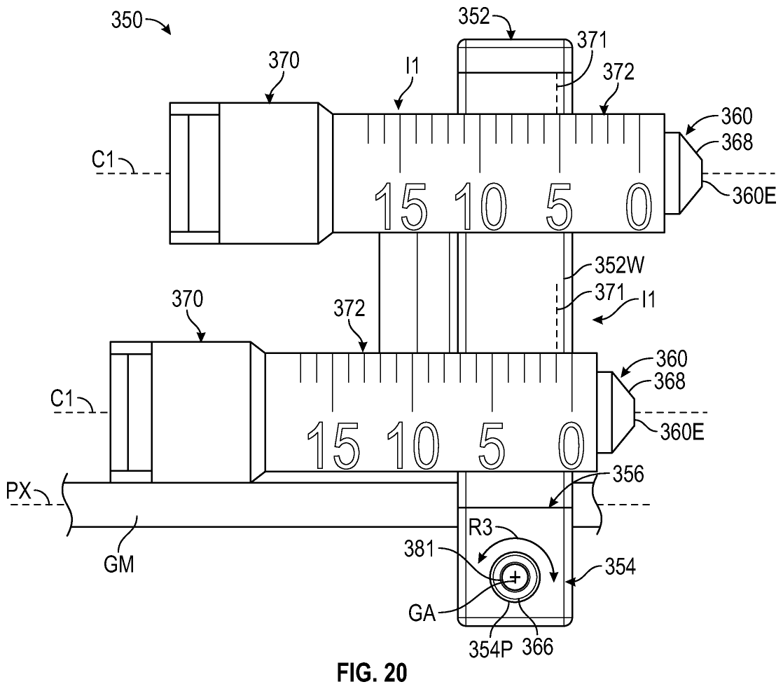

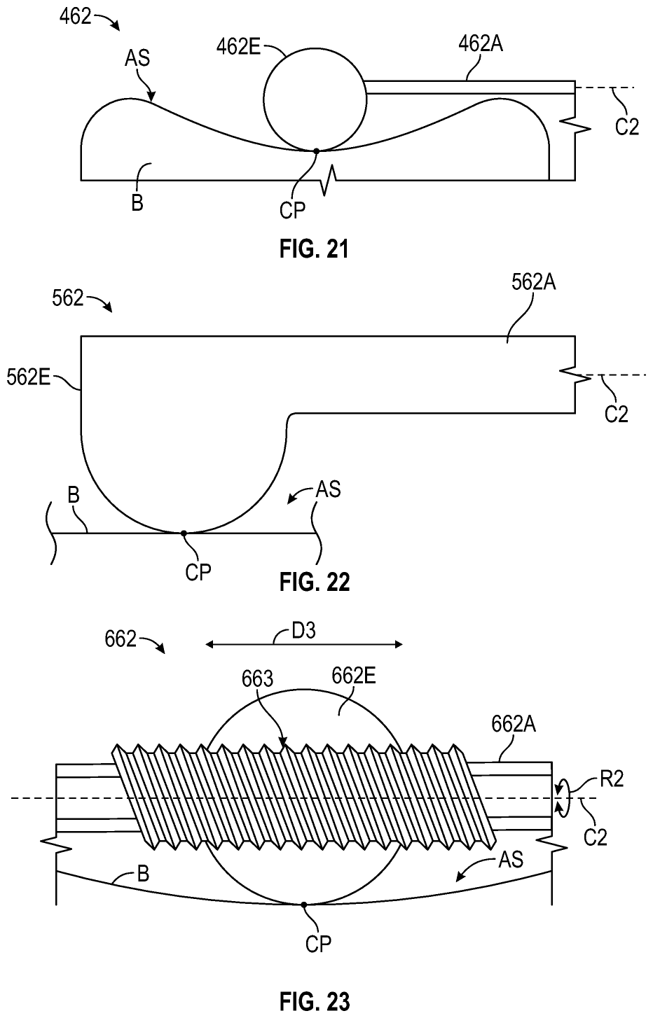

The contact members 360, 362 can have other geometries. In the implementation of FIG. 21, the engagement portion 462E has a substantially elliptical cross sectional geometry dimensioned to abut against an articular surface AS of a bone B along a respective contact point CP. The engagement portion 462E can have a substantially spherical geometry. In the implementation of FIG. 22, the engagement portion 562E can have a substantially hemispherical geometry establishing a substantially convex or arcuate contact surface. In the implementation of FIG. 23, the contact member 662 includes a main body 662A and an engagement portion 662E. The engagement portion 662E can have any of the geometries disclosed herein, such as a substantially spherical or disk shaped geometry. The main body 662A can be an elongated, threaded shaft that extends along a contact axis C2. The main body 662A and engagement portion 662E can be coupled to each other along a threaded connection 663. The contact member 662 can be configured such that the engagement portion 662E is translatable in a direction D3 along the main body 662A in response to rotation of the main body 662A in a direction R2 about the contact axis C2. Each of the contact axes C2 can be substantially perpendicular to the passage axes PX of the guide passages 358.

Still referring to FIGS. 16 and 19-20, with continuing reference to FIG. 15, the first set of contact members 360 can be arranged at various positions and/or orientations relative to each other. The contact axes C1 of the contact members 360 can be substantially parallel to each other at each position of the contact members 360 relative to the guide body 352. In other implementations, two or more of the contact axes C1 can be transverse to each other

The first set of contact members 360 may include a first pair of contact members 360, such as the contact members 360-1, 360-2, and may include a second pair of contact members 360, such as the contact members 360-3, 360-4. The guide body 352 may be configured such that the first pair of contact members 360-1, 360-2 is positioned on a first side of the reference plane REFG of the guide body 352 and such that the second pair of the contact members 360-3, 360-4 is positioned on a second side of the reference plane REFG, as illustrated in FIG. 15. The contact axes C1 of the first pair of contact members 360-1, 360-2 and/or the second pair of contact members 360-3, 360-4 can be established at substantially the same distance from the reference plane REFG. The contact axes C1 of the contact members 360-1, 360-3 can be substantially aligned relative to the guide axis X. The contact axes C1 of the contact members 360-2, 360-4 can be substantially aligned relative to the guide axis X.

Each of the first set of contact members 360 can be translatable or otherwise moveable along the first contact axis C1 to set a position of the contact member 360 relative to the guide body 352. Various techniques can be utilized to articulate the contact members 360 relative to the guide body 352. Referring to FIG. 19, with continuing reference to FIG. 15, each of the first set of contact members 360 can include a threaded guide shaft 368. The guide shaft 368 can be coupled to the guide body 352 at a threaded connection 369. Each contact member 360 can be rotatable in a direction R1 about the respective contact axis C1 to set the position of the contact member 360 relative to the guide body 352. Each of the contact axes C1 can be substantially parallel to the passage axes PX of the guide passages 358. Tooling (e.g., Allen wrench) can be utilized to engage the contact members 360 at respective interfaces 373 to rotate or otherwise move the contact members 360 (see FIGS. 14-15).

Each contact member 360 can carry a respective sheath (e.g., sleeve 370. The sheath 370 can be dimensioned to abut a sidewall 352W of the guide body 352 to limit rotation of the sheath 370 in response to translation of the respective contact member 360, as illustrated in FIG. 20. Various techniques may be utilized to secure the sheath 370 to the contact member 360. In implementations, the sheath 370 is releasably secured to a head or another portion of the contact member 360. The contact members 360 may be configured relative to respective indicia I1 (see, e.g., FIGS. 16 and 20). The indicia I1 may be associated with respective positions of the contact member 360 and contact axis CA1 relative to the guide body 352. The surgeon or assistant may move the contact members 360 relative to the indicia I1 according to one or more settings specified in a surgical plan 131 generated by the system 120 (FIG. 2).

In the implementation of FIG. 20, the indicia I1 are established by an indicator 371 relative to a ruler 372. The sheath 370 can be dimensioned to set a position of the indicator 371 relative to the ruler 372 in response to relative movement between the shaft 368 and guide body 352. The indicator 371 may be established adjacent to an edge face of the guide body 352, and the ruler 372 may be established along a periphery of the respective sheath 370, although the opposite arrangement may be utilized. The indicator 371 may be aligned with a selected position along the ruler 372 to indicate a position of the contact member 360 relative to the guide body 352.

Referring to FIGS. 16-17, with continuing reference to FIG. 15, the second set of contact members 362 can be arranged at various positions and/or orientations relative to each other. The contact axes C2 of the second set of contact members 362 can be substantially parallel to each other at each position of the contact members 362 relative to the adjustment body 354. In other implementations, two or more of the contact axes C2 can be transverse to each other. The contact axes C1, C2 of the first and second set of contact members 360, 362 can be substantially parallel to each other at each position of the contact members 360, 362. Each of the contact members 360, 362 can extend from a common side of the alignment guide 350 to establish the respective engagement portions 360E, 362E. The reference plane REFG may intersect the adjustment body 354 such that at least one or more of the contact members 362 is positioned on a first side of the reference plane REFG (e.g., contact member 362-1) and such that at least one or more other contact members 362 is positioned on a second side of the reference plane REFG (e.g., contact member 362-4) for at least a majority or all positions of the adjustment body 354 relative to the interface 356.

The second set of contact members 362 can be positioned at various distances CD from each other relative to the respective axes AA, as illustrated in FIG. 15. The distances CD between the adjacent pairs of contact members 362 of the adjustment body 354 can be the same or can differ. The set of adjustment bodies 354-1 to 354-5 (FIG. 18) can be dimensioned such that the respective distance CD between one or more adjacent pairs of the second set of contact members 362 associated with the same anatomical points of a bone of the anatomy can differ within the set of adjustment bodies 354 relative to the second direction D2 to accommodate different anatomical sizes, as illustrated by the contact members 362-3, 362-4 of the adjustment bodies 354-1 to 354-5 associated with anatomical points S2-3, S2-4 (FIG. 5). The set of adjustment bodies 354-1 to 354-5 can be dimensioned such that a length of one or more respective pairs of the slots 354S can differ within the set (e.g., slots 354S associated with the contact members 362-1) such that travel of the carriers 374 can differ relative to the direction D3 to accommodate different anatomical sizes by the surgeon.

Various techniques can be utilized to articulate each of the contact members 362 relative to the adjustment body 354. The contact members 362 can be moveable in a third direction D3 relative to the adjustment body 354. The direction D3 can be substantially parallel to the guide axis X of the guide body 352 (FIG. 15). Each of the contact members 362 can be translatable or otherwise moveable along a respective axis AA to set a position of the contact member 362 relative to the adjustment body 354.

Referring to FIG. 17, with continuing reference to FIGS. 15-16, each contact member 362 can be coupled to a respective carrier (e.g., block or traveler) 374. The contact member 362 and carrier 374 can be integrally formed or can be separate and distinct components secured to each other. Each of the contact members 362 can include an elongated main body 362A. The main body 362A can extends along a respective contact axis C2 between a first end portion 362B and a second end portion 362C. The first end portion 362B can be coupled to the carrier 374 at a fixed position relative to the contact axis C2 such that the second end portion 362C and terminal end 362T of the contact member 362 is cantilevered from the carrier 374.

Each carrier 374 can be coupled to a threaded guide shaft 376 along a threaded connection 377. The carrier 374 can cooperate with the guide shaft 376 to establish a worm drive mechanism. Opposite ends of the guide shaft 376 can be captured or otherwise supported by the adjustment body 354. The guide shaft 376 can be rotatable in the direction R2 about the respective axis AA to set the position of the contact member 362 relative to the adjustment body 354. The axis AA can be substantially parallel to the guide axis GA of the guide body 352. The carrier 374 can be translatable in the direction D3 along the guide shaft 376 to set a position of the contact member 362 relative to the adjustment body 354 in response to rotation of the guide shaft 376 in the direction R2. Tooling (e.g., Allen wrench) can be utilized to engage the guide shafts 376 at respective interfaces 375 to rotate or otherwise move the guide shafts 376 associated with the contact members 362 (see, e.g., FIGS. 14 and 16).

The guide shaft 376 can extend along a respective slot 354S established by the adjustment body 354, as illustrated in FIG. 15. Each carrier 374 can be captured in a respective slot 354S. The slot 374S can be dimensioned such that axial movement of the carrier 374 along the guide shaft 376 is bounded by opposed ends of the slot 374S. The carrier 374 can be dimensioned to abut opposed sidewalls of the slot 354S to limit rotation of the carrier 374 about the guide shaft 376.

The contact members 362 may be configured relative to respective indicia I2 (FIGS. 15 and 18). The indicia I2 may be associated with respective positions of the contact member 362 and associated contact axis CA2 relative to the adjustment body 354. The surgeon or assistant may move the contact members 362 relative to the indicia I2 according to one or more settings specified in a surgical plan 131 generated by the system 120 (FIG. 2).

In the implementation of FIG. 15, the indicia I2 are established by an indicator 378 relative to a ruler 379. The carrier 374 can be dimensioned to set a position of the indicator 378 relative to the ruler 379 in response to relative movement between the carrier 374 and adjustment body 354. The indicator 378 may be established along a face of the respective carrier 374, and the ruler 379 may be established along a portion of the adjustment body 354 adjacent to the respective slot 354S, although the opposite arrangement may be utilized. The indicator 378 may be aligned with a selected position along the ruler 379 to indicate a position of the contact member 362 relative to the alignment body 354.

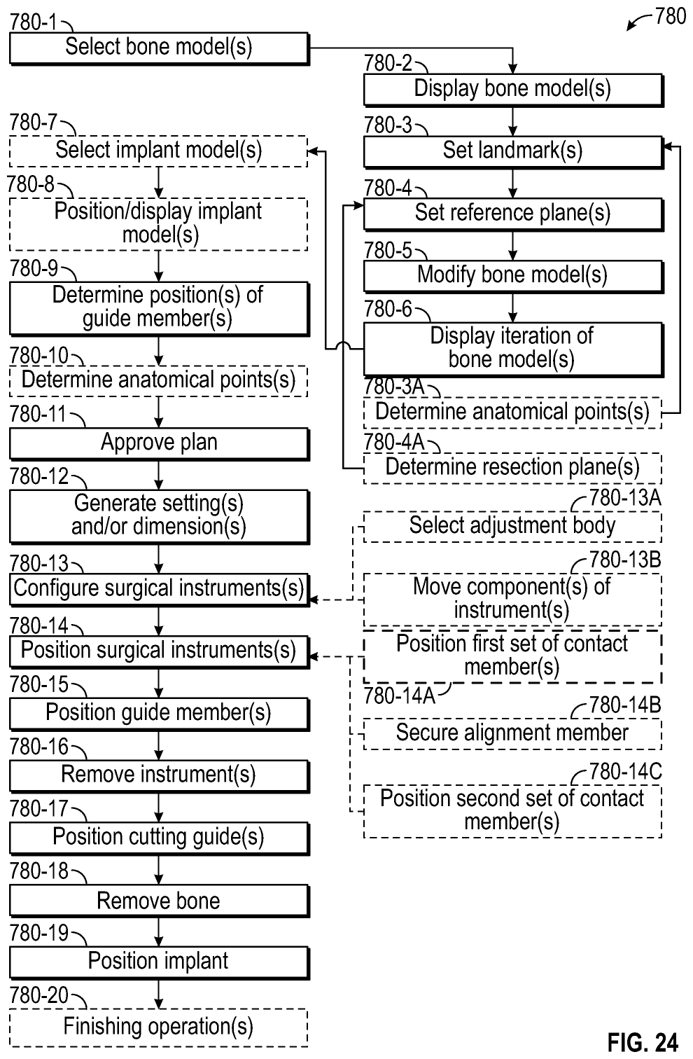

FIG. 24 illustrates an exemplary method of planning and performing an orthopaedic procedure in a flowchart 780. The method 780 may be utilized to pre-operatively plan and perform an arthroplasty for restoring functionality to ankles, shoulders, knees, hips and other joints having advanced cartilage disease. The method 780 may be utilized with any of the instrumentation and orthopedic implants disclosed herein, including the alignment guide 350. The method 780 may be utilized to configure one or more instruments for performing an orthopaedic procedure according to an associated surgical plan for a patient. Fewer or additional steps than are recited below could be performed within the scope of this disclosure, and the recited order of steps is not intended to limit this disclosure. Reference is made to the system 120 and user interface 142 for illustrative purposes.

Referring to FIGS. 2-3, with continuing reference to FIG. 24, at step 780-1 a first bone model 129-1 and/or second bone model 129-2 may be selected from a plurality of bone models 129. The models 129-1, 129-2 may be selected in response to user interaction with the menu 146M associated with the first display window 144-1 or another portion of the user interface 142. The second bone model 129-2 may be associated with a long bone such as a tibia. The first bone model 129-1 may be associated with an adjacent bone, such as a talus or an adjacent long bone.

At step 780-2, the first and second bone models 129-1, 129-2 may be displayed in the graphical user interface 142 such that a first articular surface AS1 of the first bone model 129-1 opposes and contacts a second articular surface AS2 of the second bone model 129-2, as illustrated in the first display window 144-1 of FIG. 3. The bone models 129-1, 129-2 may be displayed in the user interface 142 such that the first articular surface AS1 opposes, but is spaced apart from, the second articular surface AS2, as illustrated in the second display window 144-2.

Referring to FIG. 5, with continuing reference to FIGS. 3 and 24, at step 780-3 one or more landmarks L may be set or otherwise indicated, as illustrated in the display window 144-3. Step 780-3 can include determining and/or specifying one or more surface features SF of the respective bone, as illustrated by the saddle SF-1 extending along the non-articular surface NAS of the bone model 129-2 associated with the bone. Step 780-3 can include determining a position of the saddle SF-1 relative to the articular surface AS of the bone model 129-2 and one or more associated anatomical points P.

Step 780-3 can include determining and/or specifying positions of one or more anatomical points P along a surface of bone(s) or other tissue at the surgical site at step 780-3A. Step 780-3A can include determining and/or specifying positions of a second set of anatomical points P along the articular surface AS of the bone model 129-2 associated with the bone, as illustrated by the anatomical points S2-1 to S2-4. The anatomical points P can be determined utilizing any of the techniques disclosed herein. Step 780-3A can include determining the position of at least one of the second set of anatomical points S2-1 to S2-4, such as the anatomical point S2-1 associated with the medial maximum of the tibia, or more than one of the second set of anatomical points S2-1 to S2-4, such the anatomical point S2-3 associated with the tibial plafond. Step 780-3A can include determining the position of each of the second set of anatomical points S2-1 to S2-4. The anatomical points P and other landmarks L may be set automatically by the system 120 and/or in response to user interaction with the menu 146M associated with the display windows 144-2, 144-3, direct interaction with the display window 144-2, 144-3, and/or interaction with another portion of the user interface 142.

Referring to FIG. 6, with continuing reference to FIG. 24, at step 780-4 one or more reference planes REF may be set or otherwise indicated relative to the bone models 129-1, 129-2. The reference planes REF may be associated with respective resection planes for resecting portions of the bone(s). Step 780-4 may include determining one or more resection planes relative to an articular surface AS of a bone at step 780-4A, which may be associated with the reference planes REF adjacent to the articular surfaces AS1 and/or AS2. Step 780-4A may include setting or otherwise indicating a (e.g., first) reference plane REF (e.g., REF3 or REF4) in the user interface 142 to establish the first resection surface RS-1 of the first bone model 129-1. Step 780-4 may include setting or otherwise indicating a (e.g., second) reference plane REF (e.g., REF1 or REF2) in the user interface 142 to establish the second resection surface RS-2 of the second bone model 129-2. The reference planes REF may be set in response to user interaction with the menu 146M associated with the display window 144-4, direct interaction with the display window 144-4, and/or interaction with another portion of the user interface 142. The user may specify an offset distance or resection depth utilizing the entry field 146E.

At step 780-5, one or more of the bone models 129-1, 129-2 may be modified. The modification may be applied to a local copy of the bone models 129-1, 129-2 and/or a global copy of the bone models 129-1, 129-2 in the database 128. Step 780-5 may include generating one or more iterations of first and second (e.g., resection) bone models 129-1′, 129-2′. The bone models 129-1′, 129-2′ may exclude respective volumes of the bone models 129-1, 129-2 between the reference planes REF and articular surfaces AS1, AS2 of the bone models 129-1, 129-2, as illustrated in the fifth display window 144-5. The first resection surface RS-1 of the first bone model 129-1′ may be positioned in contact with the second resection surface RS-2 of the second bone model 129-2′ to establish a contact region CR. Step 780-5 may include one or more iterations of moving the bone models 129-1′, 129-2′ relative to each other to establish the contact region CR, which may occur automatically or in response to user interaction with the user interface 142. At step 274-6, portions of the bone models 129-1′, 129-2′ along the contact region CR may be displayed in the display window 144-5 or another portion of the graphical user interface 142.

Referring to FIG. 7, with continuing reference to FIG. 24, at step 780-7 one or more implant models 130 may be selected from a plurality of implant models 130. The implant model(s) 130 may be selected automatically based on various attributes of the patient including the selected bone models 129 and/or in response to user interaction with the menu 146M or another portion of the user interface 142.

At step 780-8, the selected implant model(s) 130 may be positioned relative to the first and/or second bone models 129-1′, 129-2′, which may occur subsequent to modifying the bone models 129-1 and/or 129-2 and establishing the contact region CR at step 780-5. The selected implant model(s) 130 may be displayed in the user interface 142. One or more iterations of moving the bone models 129-1′, 129-2′ relative to each other at step 780-5 may occur prior to, during and/or subsequent to positioning the implant model(s) 130 at step 780-8.

Referring to FIGS. 8-10, with continuing reference to FIG. 24, at step 780-9, the method 780 may include determining position(s) of one or more guide member(s) GM relative to the bone models 129-1′, 129-2′. The guide members GM can include any of the guide members disclosed herein, such as an elongated guide pin. Step 780-9 can include determining an orientation of at least one guide member GM relative to the respective reference plane REF, which can be associated with a respective resection plane. The orientation can include an insertion point and/or trajectory T of the guide member GM. The trajectories T may be associated with positions along the respective bone model 129-1′, 129-2′ relative to one or more landmarks L specified in the surgical plan 131, including any of the anatomical points P (FIG. 5, see also FIG. 11). The trajectories T may be determined based on a geometry of a surgical instrument associated with the guide members GM and/or position of the surgical instrument relative to the bone model 129 specified in the surgical plan 131. The instrument can include any of the alignment guides and guide passage arrangements disclosed herein.

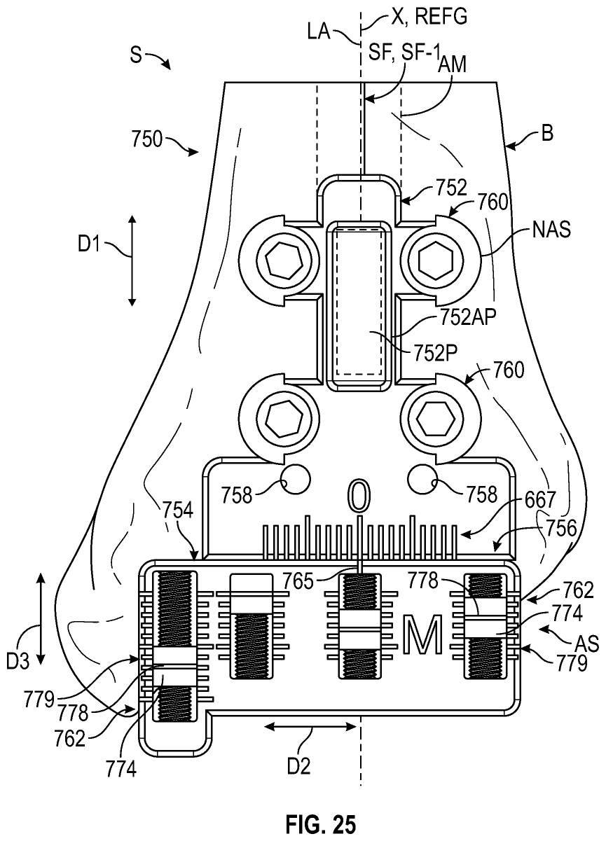

Referring to FIG. 11, with continuing reference to FIG. 24, at step 780-10 positions of one or more anatomical points P along a surface of bone(s) or other tissue at the surgical site may be determined or specified. Step 780-10 can include determining and/or specifying positions of a first set of anatomical points P along a non-articular surface NAS of the respective bone, as illustrated by the first set of anatomical points S1-1 to S1-4. The anatomical points P can be determined utilizing any of the techniques disclosed herein. The anatomical points P may be set automatically by the system 120 and/or in response to user interaction with a menu 146M associated with a respective display window 144 such as the display window 144-3 (FIG. 5), direct interaction with the display window 144 and/or another portion of the user interface 142. The first set of anatomical points S1-1 to S1-4 may be determined and set independently. One or more of the anatomical points P may be constrained relative to each other. In implementations, the anatomical points S1-1 to S1-4 may be grouped together and moveable as a group GP (shown in dashed lines in FIG. 5 for illustrative purposes) to set the positions of the anatomical points S1-1 to S1-4. The group GP may be established based on a geometry of the selected alignment guide and dimensions of the associated guide passages. For example, the group GP may be established by the positions of the guide passages 758 relative to each other and along the alignment guide 750 of FIG. 25. The alignment guide 750 may incorporate the features of the alignment guide 350.

At step 780-11, the surgeon or another user can approve a surgical plan 131 (FIG. 2). Approving the surgical plan 131 may occur automatically or in response to interaction with the user interface 142. The surgical plan 131 can include any of the parameters established in the method 780, including the anatomical points P and other landmarks L associated with the bone models 129-1, 129-2, such as the surface features SF, first and/or second sets of anatomical points S1, S2, and the specified reference planes REF associated with the resection surfaces RS-1, RS-2. The surgical plan 131 can include other parameters, including the selected bone models 129-1/129-1′/129-1′ and/or selected implant model(s) 130.

The method 780 may include one or more steps to implement each surgical plan 131. At step 780-12, one or more settings and/or dimensions for surgical instrument(s) may be generated. The setting(s) and dimension(s) may be based on trajectories associated with one or more bone models, such as the trajectories T1, T2, T3 and/or T4 associated with the bone models 129-1′, 129-2′ of FIGS. 8-10. The surgical instruments can include any of the alignment guides disclosed herein, such as the alignment guide 350 or alignment guide 750. The settings generated at step 780-12 can be stored in a surgical plan 131 (FIG. 2). Exemplary settings include patient information, procedure type, implant type and position, fastener sizes and lengths, resection depths and angles, alignment guide and/or other surgical instrument positions, including contact member positions relative to the surgical site, guide member trajectories and insertion points, etc.

At step 780-13, one or more surgical instruments can be configured according to the setting(s) and dimension(s) generated at step 780-12. Step 780-13 may include configuring the surgical instrument(s) according to any of the techniques disclosed herein. Step 780-13 may include fabricating surgical instrument(s) according to the dimension(s) generated at step 780-12. The fabricated instrument(s) may include one or more patient-specific surfaces or components.

Referring to FIG. 25, with continuing reference to FIG. 24, step 780-13 can include configuring the alignment guide 750 or another surgical instrument according to the setting(s) generated at step 780-12. One or more of the settings can be transferred by the surgeon or assistant to the alignment guide 750 or other surgical instruments. Step 780-13 may include assembling the alignment guide 750. Step 780-13 may include selecting the adjustment body 754 from a set of adjustment bodies (see, e.g., FIG. 18) at step 780-13A. The surgeon or assistant can select the adjustment body 754 based on an anatomy of the patient. The set of adjustment bodies, including the selected adjustment body 754, can be dimensioned according to any of the techniques disclosed herein, can be provided as a kit to the surgeon, and can be interchangeably mountable to the guide body 752 along the interface 756. Step 780-13A may include selecting the adjustment body 754 from a set of adjustment bodies based on the positions of the first set of anatomical points S1-1 to S1-4 determined at step 780-12 and/or the second sets of anatomical points S2-1 to S2-4 determined at step 780-3A.

Step 780-13 may include moving or otherwise configuring component(s) of the alignment guide 750 at step 780-13B according to the setting(s) generated at step 780-12. Step 780-13B may occur prior, during and/or subsequent to assembling the alignment guide 750.

Step 780-13B may include setting a position of the guide body 752 and selected adjustment body 754 relative to each other along the interface 756. Step 780-13B may include setting the position(s) of the bodies 752, 754 based on the positions of the anatomical points P determined at steps 780-3A and/or 780-10, such as the first set of anatomical points S1-1 to S1-4 and/or second sets of anatomical points S2-1 to S2-4. Setting the position of the adjustment body 754 may include moving the adjustment body 754 and guide body 752 in the direction D2 relative to each other along the interface 756 subsequent to selecting the adjustment body 754 at step 780-13A and securing the selected adjustment body 754 to the guide body 752. Step 780-13B may include setting a position of the indicator 765 relative to the ruler 765 according to a setting determined at step 780-12, which can be specified in a surgical plan 131.

Step 780-13B can include setting a position of each of the first and/or second sets of contact members 760, 762 based on the orientations of the guide members GM determined at step 780-9. Step 780-13B may include setting the position of each of the first set of contact members 760 based on the positions of the anatomical point(s) P determined at step 780-10. Step 780-13B may include setting the position of each of the second set of contact members 762 based on the positions of the anatomical point(s) P determined at step 780-3A. Step 780-13B may occur subsequent to selecting the adjustment body 754 at step 780-13A and then securing the selected adjustment body 754 to the guide body 752. Step 780-13B may include setting position of the indicators 778 relative to the rulers 769 associated with the contact members 762 (FIG. 25) and/or setting positions of the indicators 771 relative to the rulers 772 associated with the contact members 760 (FIG. 26) according to respective settings determined at step 780-12, which can be specified in the surgical plan 131.

In implementations, the bone B can be a long bone such as a tibia. Determining the positions of the second set of anatomical points S2-1 to S2-4 at step 780-3A can include identifying the positions of the second set of anatomical points S2-1 to S2-4 in a silhouette of the articular surface AS of the tibia, as illustrated in FIG. 11. The second set of anatomical points S2-1 to S2-4 can be distributed in a medial-lateral M/L direction of the tibia (FIG. 11). The silhouette may be depicted in one or more preoperative and/or intraoperative images. The second set of anatomical points S2-1 to S2-4 may be associated with the medial maximum, tibia groove, tibial plafond and/or lateral maximum of the articular surface AS of the tibia.

At step 780-14, the surgical instrument(s) may be positioned relative to the bone B at the surgical site S according to the setting(s) generated at step 780-12. The alignment guide 750 may be positioned relative to the bone B in a configured state according to the settings implemented at step 780-13. The surgeon or assistant may adjust one or more settings of the alignment guide 750 subsequent to positioning the alignment guide 750 in contact with the bone B, including the position of the adjustment body 754 relative to the guide body 752 and/or the positions of any of the contact members 760, 762.

Step 780-14 may include positioning the first and second sets of contact members 760, 762 in contact with the bone B along respective contact (e.g., anatomical or touch) points CP, as illustrated in FIGS. 26-28. Step 780-14 can include positioning the first set of contact members 760 to establish contact at respective contact points CP along a non-articular surface NAS of the bone B at step 780-14A, as illustrated by the contact points CP-1 to CP4 in FIG. 26. The contact points CP-1 to CP4 may be associated with the first set S1 of anatomical points S1-1 to S1-4 along the non-articular surface NAS of the bone model 129-2 (FIG. 5). Step 780-14 can occur such that the positions of the contact points CP-1 to CP4 substantially correspond to the positions of the first set S1 of anatomical points S1-1 to S1-4 specified in the surgical plan 131.

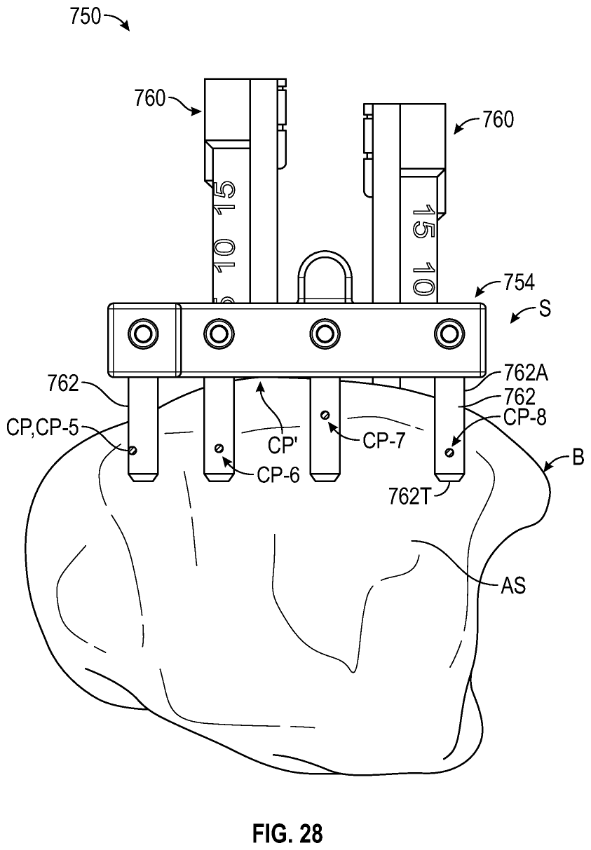

The alignment guide 750 can be positioned such that the first set S1 of contact points CP-1 to CP-4 may be distributed on opposite sides of a surface features SF, such as a saddle SF-1 of the bone B. The contact members 760 can be arranged to straddle a length of the saddle SF-1. Step 780-14A may include identifying the position of each, or at least some, of the contact points CP along the bone B associated with the anatomical points P of the respective bone model 129, 129′ specified in the surgical plan 131, such as the first set of anatomical points S1-1 to S1-4 (see also FIG. 5). Positioning the first set of contact members 760 can occur such that the reference plane REFG of the guide body 752 is substantially parallel to a longitudinal axis LA of the bone B, as illustrated in FIGS. 26 and 29. Step 780-14A can occur such that a surface of the guide body 752 and/or adjustment body 754 rests on or otherwise contacts a surface of the bone B, such as the saddle SF-1 (see, e.g., contact point of FIG. 28), which may serve to increase stability in positioning of the alignment guide 750. In other implementations, step 780-14 can occur such that the guide body 752 and adjustment body 754 are spaced apart from the adjacent bone B.

Step 780-14 can include securing an alignment member AM in a passage 752P of the guide body 752 (shown in dashed lines for illustrative purposes, see also FIG. 29) at step 780-14B. Step 780-14 may occur such that the alignment member AM may be substantially parallel to a guide axis X of the guide body 752. Positioning the first set of contact members 760 at step 780-14A may include substantially aligning the alignment member AM relative to a landmark of the bone B, such as the longitudinal axis LA of the bone B.

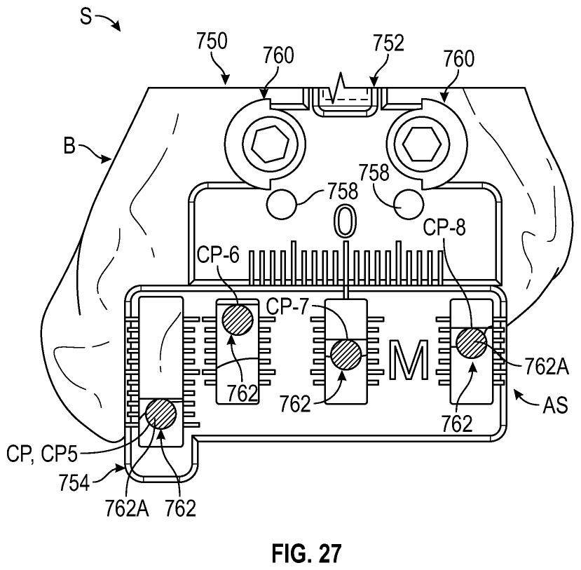

Referring to FIGS. 27-28, with continuing reference to FIG. 24, step 780-14 may include positioning the second set of contact members 762 along respective contact points CP at step 780-14C. Step 780-14C can include positioning the second set of contact members 762 to establish contact at respective contact points CP along an articular surface AS of the bone B, as illustrated by contact points CP-5 to CP8. The carriers 774 of the respective contact members 762 are omitted from FIG. 27 for illustrative purposes. Step 780-14 can occur such that the positions of the contact points CP-1 to CP4 substantially correspond to the positions of the first set S1 of anatomical points S1-1 to S1-4 specified in the surgical plan 131. The contact points CP-5 to CP-8 may be associated with the second set S2 of anatomical points S2-1 to S2-4 along the articular surface AS2 of the bone model 129-2 (FIG. 5).

Various techniques can be utilized to position the contact members 762. In implementations, positioning the contact members 762 in contact with the respective contact points CP-5 to CP-8 along the articular surface AS2 can occur in sequence beginning with the contact member 762-1 and ending with the contact member 762-4, which may improve repeatability and precision in placement of the alignment guide 750 according to the surgical plan 131. Positioning the second set of contact members 762 at step 780-14C may occur subsequent to positioning the first set of contact members 760 at step 780-14A. Previously positioning the first set of contact members 760 at step 780-14A can limit the degrees of freedom of the second set of contact members 762 (e.g., 1 degree of freedom), which can improve precision in placement of the alignment guide 750 and associated guide members GM according to the surgical plan 131. In other implementations, positioning the second set of contact members 762 at step 780-14C may occur prior to, or concurrently with, positioning the first set of contact members 760 at step 780-14A. Step 780-14C may include identifying the position of each, or at least some, of the contact points CP along the bone B associated with the anatomical points P of the second set of anatomical points S2-1 to S2-4 associated with the respective bone model 129, 129′ specified in the surgical plan 131 (see FIG. 5).

Referring to FIG. 29, with continuing reference to FIG. 24, one or more guide members GM may be positioned with the alignment guide 750 at step 780-15. Step 780-15 can include inserting at least one guide member GM through a respective guide passage 758 and then into the bone B, which may occur subsequent to positioning the contact members 760, 762 of the alignment guide 750 in abutment with the bone B at step 780-14. Each guide member GM may be moved at least partially through the passage 758 and along the respective passage axis PX to establish the determined trajectory of the guide member GM. The guide members GM may be oriented according to the associated trajectories (e.g., trajectories T3, T4 of FIGS. 8-9) specified by one or more parameters in the surgical plan 131. Step 780-15 may occur such that the guide members GM are substantially parallel to each other. The alignment guide 750 may be configured such that the trajectories and insertion points of the guide members GM are established relative to the bone B to ensure a resection of the bone B substantially conforms to the resection plane(s) specified in the surgical plan 131 (see, e.g., reference planes REF of FIG. 6).

At step 780-16, the alignment guide 750 and other instrument(s) may be removed from the guide members GM and from the surgical site S. The guide members GM can remain in the bone B to position one or more surgical instruments relative to the surgical site S.

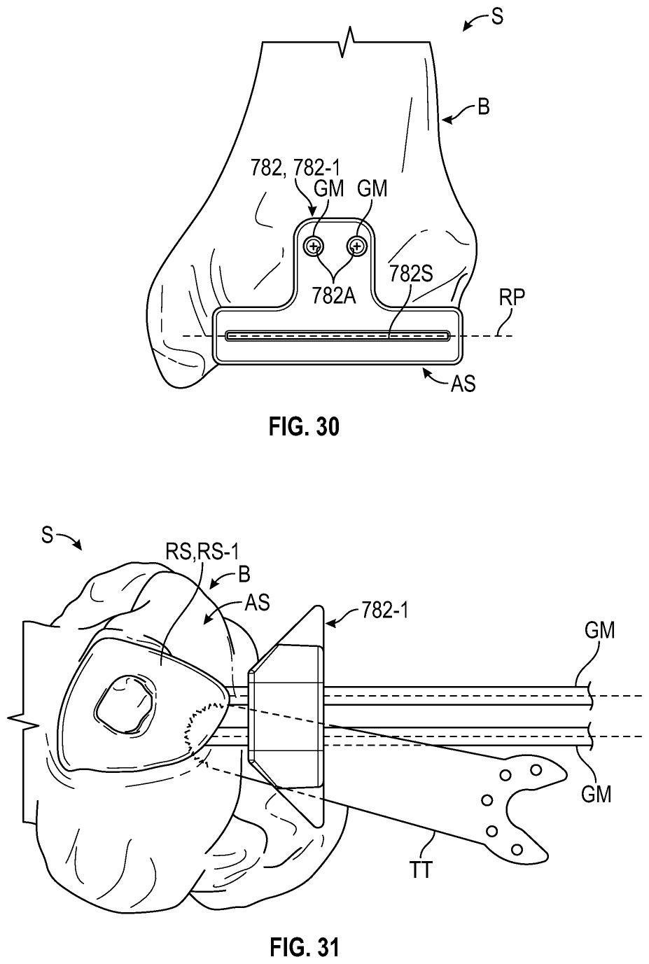

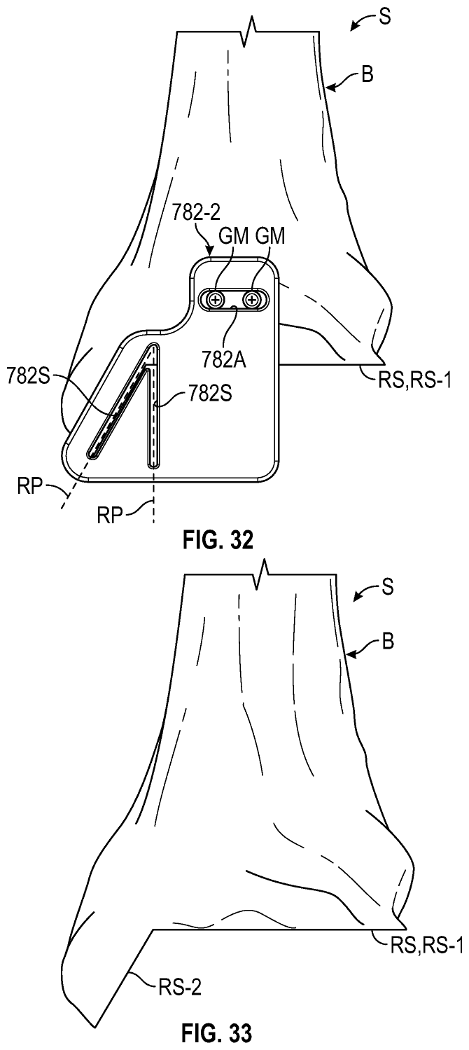

Referring to FIGS. 30-31, with continuing reference to FIG. 24, at step 780-17 one or more cutting guides (e.g., jigs) 782 may be positioned adjacent to the bone B relative to the surgical site S. The cutting guides 782 can include a first cutting guide 782-1 and a second cutting guide 782-2 (FIG. 32). Each cutting guide 782 may be positioned relative to the bone B according to the trajectories of the respective guide member(s) GM. Each cutting guide 782 can be a resection guide having one or more resection slots 782S, which can extend along a respective resection plane RP (shown in dashed lines in FIG. 30). Each resection slot 782S can be dimensioned to receive and guide an orientation of tooling TT such as a saw blade (FIG. 31).

Step 780-17 may include positioning the first cutting guide 782-1 along at least one or more of the guide members GM. The resection plane RP of the first cutting guide 782-1 may be associated with one of the specified reference planes REF of the respective bone model 129, such as one of the reference planes REF1, REF2 associated with the bone model 129-2 (FIG. 6). Step 780-17 can occur such that an orientation of the resection slot 782S and associated resection plane RP substantially corresponds to the determined reference plane REF specified in the surgical plan 131. The cutting guide 782-1 can include one or more apertures 782A that receive one or more of the guide members GM. The apertures 782A can be dimensioned according to predetermined offsets from the contact members 760, 762 to position the slot 782S and resection plane RP relative to the bone B according to the surgical plan 131. The planning system 120 can be configured to store the relative dimensions of the aperture(s) 782A and associated slot(s) 782S of the cutting guides 782. The settings of the alignment guide 750 and other instruments can be generated at step 780-12 based on the relative dimensions such that the resection plane(s) RP substantially correspond to respective reference plane(s) REF specified in the surgical plan 131 (see, e.g., FIGS. 2 and 5-6).

At step 780-18, a portion of the bone B may be resected along the resection plane RP or otherwise removed to establish a resection surface RS of the bone B, as illustrated by the resection surface RS-1 in FIGS. 31-32. Step 780-19 can include moving a cutting instrument such as the tooling TT through the slot 782S to resect or otherwise remove a portion of the bone B.

Referring to FIG. 32, with continuing reference to FIG. 24, the second cutting guide 782-2 may be positioned along the guide members GM, which may occur subsequent to removing the first cutting guide 782-1. The aperture 782S of the cutting guide 782-2 may be a slot 782S dimensioned to receive two or more of the guide members GM for facilitating lateral movement of the second cutting guide 782-2. Another portion of the bone B may be resected along the resection plane RP to establish another resection surface RS-2 of the bone B. The resection surface RS-2 may be transverse to the resection surface RS-1 established with the first cutting guide 782-1, as illustrated in FIG. 33. The bone B may be a tibia, and the transverse cut established by the second resection surface RS-2 may establish a “gutter cut” of a medial malleolus of the bone B. The resection plane RP may be associated with one of the specified reference planes REF of the respective bone model 129, such as the reference plane REF5 associated with the bone model 129-2 (FIG. 6).

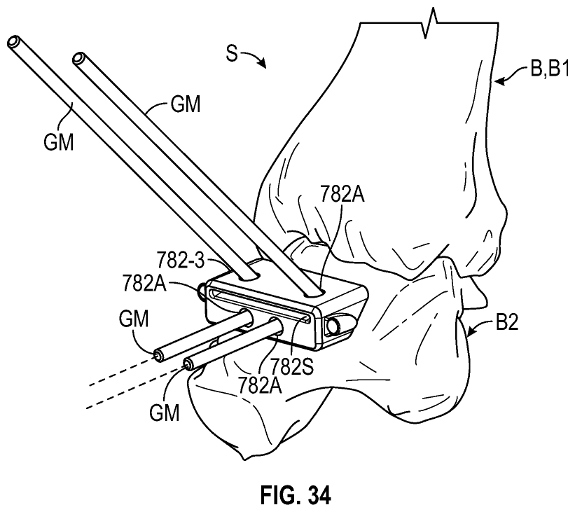

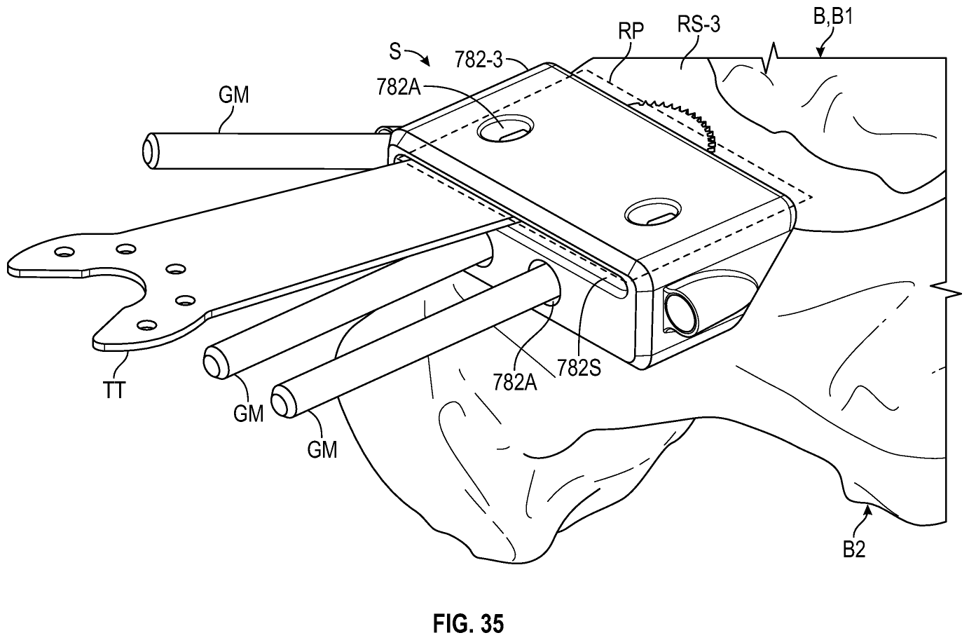

Referring to FIGS. 34-35, with continuing reference to FIG. 24, the bone B may be a first bone B1 adjacent to a second bone B2. Articular surfaces of the bones B1, B2 may cooperate to establish a joint, including any of the joints disclosed herein such as an ankle joint. The first bone B1 may be a tibia. The second bone B2 may be a talus.

Step 780-17 may include positioning a third cutting guide 782-3 relative to the second bone B2 at the surgical site S. The cutting guide 782-3 may be positioned according to the trajectories of one or more the guide members GM. Step 780-17 may include positioning the cutting guide 782-3 along the guide members GM. The guide members GM may be positioned according to any of the techniques disclosed herein, including with the alignment guide 750.

The cutting guide 782-3 can include at least one slot 782S dimensioned to receive tooling TT such as a saw (FIG. 35). The cutting guide 782-3 may establish at least one resection plane RP (shown in dashed lines in FIG. 35). The resection plane RP may be associated with one of the reference planes REF of the respective bone model 129, such as the reference plane REF3 or REF4 associated with the bone model 129-1 (FIG. 6). The resection plane RP may be oriented according to the trajectories of the guide member(s) GM. At step 780-18, a portion of the bone B2 may be resected along the resection plane RP to establish a resection surface RS-3 of the bone B2, as illustrated in FIG. 35.

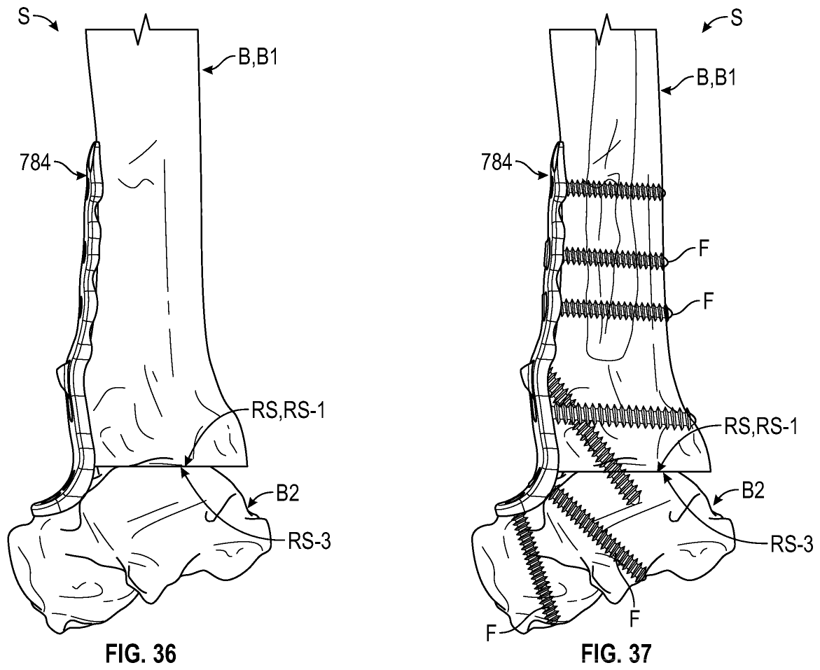

Referring to FIGS. 36-37, with continuing reference to FIG. 24, the cutting guide(s) 778 can be removed from the surgical site S. At step 780-19, the resection surfaces RS-1, RS-2 of the first and second bones B1, B2 may be moved into abutment to promote fusion of the resected surfaces RS-1, RS-2. Step 780-19 may include positioning the bones B1, B2 to establish a contact area that substantially corresponds to the contact region CR of the associated surgical plan 131 (see, e.g., FIGS. 2 and 6). The surgeon may adjust the relative position between the resection surfaces RS-1, RS-2 of the bones B1, B2.

At least one implant 784 may be positioned relative to the bones B1, B2. The implant 784 may be associated with an implant model 130 of the surgical plan 131 (see, e.g., FIGS. 2 and 7). Step 780-19 may occur subsequent to moving the resection surfaces RS-1, RS-2 of the bones B1, B2 into abutment. The implant 784 may be positioned to span across an interface between the bones B1, B2.

Various techniques may be utilized to secure the implant 784 to the bones B1, B2, including one or more fasteners F (FIG. 37). Exemplary fasteners can include nails, pins, locking and/or non-locking compression screws, suture, etc. At step 780-20, one or more finishing operations may be performed. Exemplary finishing operations may include closing an incision at the surgical site S.

The novel devices and methods of this disclosure can be utilized to precisely place one or more guide members for guiding placement of surgical instruments relative to a surgical site. The disclosed surgical instruments may be reusable and may be configured according to settings established in a surgical plan. The surgeon or assistant may configure the alignment guide based on the settings. The settings may include specified positions of contact members of the alignment guide for facilitating contact with bone or other tissue at contact points that may substantially correspond to respective anatomical points or other landmarks specified in the surgical plan. The techniques disclosed herein can improve precision in placement of the respective guide members for improving accuracy in forming resection surfaces according to the surgical plan established for the patient. The disclosed systems and methods can be utilized to reduce operative time and complexity.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. An alignment guide assembly for an orthopaedic procedure comprising:
a guide body carrying a first set of adjustable contact members dimensioned to contact bone at respective contact points, the guide body including at least one guide passage dimensioned to set a trajectory of a guide member relative to bone; and
at least one adjustment body coupled to the guide body along an interface, the at least one adjustment body carrying a second set of adjustable contact members dimensioned to contact bone at respective contact points, each contact member of the second set of contact members coupled to a respective carrier, and the carrier translatable along a guide shaft to set a position of the respective contact member relative to the at least one adjustment body in response to rotation of the guide shaft.

2. The assembly as recited in claim 1, wherein:
the second set of contact members are distributed in a first direction along the at least one adjustment body;
the at least one adjustment body includes a set of adjustment bodies, the adjustment bodies interchangeably mountable to the guide body along the interface; and
the adjustment bodies are dimensioned such that a distance between one or more adjacent pairs of the second set of contact members differ within the set of adjustment bodies relative to the first direction.

3. The assembly as recited in claim 1, wherein:
the guide shaft extends along a slot of the at least one adjustment body; and
the carrier is captured in the respective slot, axial movement of the carrier along the guide shaft is bounded by opposed ends of the slot, and the carrier abuts opposed sidewalls of the slot to limit rotation of the carrier about the guide shaft.

4. The assembly as recited in claim 1, wherein:
each of the first set of contact members is a threaded shaft coupled to the guide body at a threaded connection; and
each of the first set of contact members carries a respective sheath dimensioned to abut a sidewall of the guide body, and the sheath is dimensioned to set a position of an indicator relative to a ruler in response to relative movement between the threaded shaft and the guide body.

5. The assembly as recited in claim 1, wherein:
the first set of contact members are dimensioned such that the contact points are established at a free end of the respective contact members; and
the second set of contact members are dimensioned such that the contact points are spaced apart from a free end of the respective contact members.

6. The assembly as recited in claim 1, wherein each contact member of the first and second sets of contact members includes an elongated body extending along a respective contact axis, the contact axes of the first set of contact members are substantially parallel to each other, and the contact axes of the second set of contact members are substantially parallel to each other.

7-9. (canceled)

10. The assembly as recited in claim 2, wherein a total number of the contact members of the second set of contact members is equal for the set of adjustment bodies.

11-14. (canceled)

15. A method of performing an orthopaedic procedure comprising:
determining a resection plane relative to an articular surface of a bone;
determining an orientation of at least one guide member relative to the resection plane;
determining positions of a first set of anatomical points along a non-articular surface of the bone;
determining positions of a second set of anatomical points along the articular surface of the bone;
configuring an alignment guide, wherein:
the alignment guide includes a guide body coupled to at least one adjustment body along an interface extending in a first direction, the guide body includes a guide passage, the guide body carries a first set of adjustable contact members, and the at least one adjustment body carries a second set of adjustable contact members distributed in the first direction; and
the configuring step includes setting a position of each contact member of the first and second sets of contact members based on the determined orientation, setting the position of each contact member of the second set of contact members based on the determined positions of the second set of anatomical points, and setting a position of the at least one adjustment body and the guide body relative to each other along the interface based on the determined positions of the first and second sets of anatomical points; and
positioning the alignment guide relative to the bone according to the set positions such that the second set of contact members contact the respective anatomical points of the second set of anatomical points, and then inserting the at least one guide member through the guide passage and into the bone.

16. The method as recited in claim 15, wherein the positioning step occurs such that the first set of contact members establish contact along respective contact points of a non-articular surface of the bone.

17. The method as recited in claim 15, further comprising:
positioning a resection guide along the at least one guide member such that an orientation of a resection slot of the resection guide substantially corresponds to the determined resection plane; and
moving a cutting instrument through the resection slot to resect a portion of the bone.

18. The method as recited in claim 15, wherein the bone is a tibia, the step of determining the positions of the second set of anatomical points includes identifying the positions of the second set of anatomical points in a silhouette of the articular surface of the tibia, and the second set of anatomical points are distributed in a medial-lateral direction of the tibia.

19. The method as recited in claim 18, wherein the second set of anatomical points include a medial maximum and a tibial plafond of the articular surface of the tibia.

20. The method as recited in claim 19, wherein the second set of anatomical points include a tibial groove and a lateral maximum of the articular surface of the tibia.

21. The method as recited in claim 15, wherein the step of setting the position of the at least one adjustment body includes moving the at least one adjustment body and the guide body in the first direction relative to each other along the interface.

22. The method as recited in claim 15, wherein:
the at least one adjustment body includes a set of adjustment bodies, the adjustment bodies are interchangeably mountable to the guide body along the interface, and the adjustment bodies are dimensioned such that a distance between one or more adjacent pairs of the second set of contact members associated with the same anatomical points of the second set of anatomical points of the bone differs within the set of adjustment bodies relative to the first direction; and
the step of configuring the alignment guide includes selecting the at least one adjustment body from the set of adjustment bodies based on the determined positions of the first and second sets of anatomical points, and the step of setting the position of each of the second set of contact members occurs subsequent to the selecting step.

23-31. (canceled)

32. A system for performing an orthopaedic procedure comprising:
processing circuitry coupled to memory;
wherein the processing circuitry is operable to execute a planning environment, and the planning environment is operable to:
determine a resection plane relative to an articular surface of a bone;
determine an orientation of at least one guide element relative to the resection plane;
determine positions of a first set of anatomical points along the articular surface of the bone based on a silhouette of the articular surface and the determined orientation of the at least one guide element; and
generate one or more settings for configuring an alignment guide, wherein:
the alignment guide includes a guide passage for receiving the at least one guide element and carries a first set of adjustable contact members; and
the one or more settings establish a position of each contact member of the first set of contact members based on the determined positions of the first set of anatomical points.

33. The system as recited in claim 32, wherein:
the bone is a tibia; and
the silhouette is associated with the articular surface of the tibia, and the first set of anatomical points are distributed in a medial-lateral direction of the tibia.

34. The system as recited in claim 33, wherein the first set of anatomical points include a medial maximum and a tibial plafond of the articular surface of the tibia.

35. The system as recited in claim 32, wherein:
the alignment guide includes a guide body coupled to at least one adjustment body along an interface;
the at least one adjustment body carries the first set of adjustable contact members;
the guide body includes the guide passage; and
the one or more settings establish a position of the at least one adjustment body and the guide body relative to each other along the interface based on the determined positions of the first set of anatomical points.

36. The system as recited in claim 35, wherein:
the planning environment is operable to determine positions of a second set of anatomical points along a non-articular surface of the bone;
the guide body carries a second set of adjustable contact members; and
the one or more settings establish the position of the at least one adjustment body and the guide body relative to each other along the interface based on the determined positions of the second set of anatomical points.

37. The system as recited in claim 35, wherein:
the at least one adjustment body includes a set of adjustment bodies, the adjustment bodies are interchangeably mountable to the guide body along the interface extending in a first direction, and the adjustment bodies are dimensioned such that a distance between one or more adjacent pairs of the first set of contact members differs within the set of adjustment bodies relative to the first direction.

38. The system as recited in claim 37, wherein the one or more settings includes a selection of the at least one adjustment body from the set of adjustment bodies based on the determined positions of the first set of anatomical points.

39. An alignment guide assembly for an orthopaedic procedure comprising:
a guide body including at least one guide passage dimensioned to set a trajectory of a guide element relative to bone; and
a set of adjustment bodies including first and second adjustment bodies interchangeably mountable to the guide body along an interface, each of the adjustment bodies including a set of adjustable contact members dimensioned to contact bone at respective contact points, the contact members moveable in a first direction relative to the respective adjustment body, and a distribution of the set of contact members of the first adjustment body differs from a distribution of the set of contact members of the second adjustment body relative to a second direction transverse to the first direction.

40. The assembly as recited in claim 39, wherein:
the guide body and the respective adjustment body are moveable relative to each other along the interface with respect to the second direction.

41. The assembly as recited in claim 39, wherein:
a total number of contact members in the set of contact members is equal for the first and second adjustment bodies.

42. The assembly as recited in claim 39, wherein:
each contact member is coupled to a respective carrier, and the carrier is coupled to the respective adjustment body; and
the first and second adjustment bodies are dimensioned such that a travel of one or more of the respective carriers associated with the first adjustment body differs from a travel of one or more of the respective carriers associated with the second adjustment body relative to the first direction.
